SEQUENCE LISTING (34 sequences)

SEQ ID NO: 1 - DNA, 2322 bp, Glycine max
  CDS (370)...(1620)
agcccaagcccccttcccagctggttcaaaaactccccttcctcaaactcaaaccctagcagcgtcagctcaacacccttgcggatcttcaagcgccccttccctccgccctctccggccaagcacattcgcgcgctgctcgcccgccgccacggttccgtcaagccgaacgaagcctccataccggaggccagcgagtgtgagctcggcctcgacaagagctttggctttgctaagcagttttcggctcattatgagctcagtgacgaagtcggccgggggcattttgggtatacctgctccgctaaaggcaagaaaggggcgttcaagggcttaaatgttgctgtcaaagtcattcctaaagccaagatgaccacagcaattgctatagaggatgtaaggagagaagtgaagatattgagggctttaacaggacataagaatctggtgcaattctatgaagcctatgaagatgatgacaatgtttatatagttatggagttgtgcaaaggaggggaattgctagataggattctttccaggggtggaaagtactcagaagaggatgccagagtagttatgatccaaatattgagtgtggtagctttttgtcatctgcagggtgttgttcaccgtgatctcaagccagagaattttcttttcacttctaaggatgacaagtccacgctgaaggccattgattttgggttgtctgactatgtaaagccagatgagaggttgaatgatattgtgggaagtgcttattatgtagctccagaagttttgcatagatcttatgggacagaggcagatatgtggagcattggtgtaattgcttatattcttttatgcggaagccgtcccttttgggcccggacagaatctggtatatttcgggctgtactgaaggcagatccaagttttgatgaggctccttggccttctttatcggttgatgccaaagattttgtaaagaggttgttgaataaggattatcgtaaaagattgactgcggctcaggcactaagtcatccatggctggtgaatcatcatgatgatatgaggatacctttggatatgataatccacaagcttgttaaagcttacatttgctcgtcttcgttgcgcaaatctgctttacgggctcttgcaaagacattaacagtagctcagctagcttatctcagagatcaatttactctgttagggccaaacaaaagtggattaatttctatgcagaactttaagacggctgttttgaggagctctacagatgcctcaaaggattcacgggtcttagattatgtcagtatggttagttctatccaatataggaaattagattttgaggagttttgtgctgctgctataagtgtgcaccaactcgagggaatggagacctgggagcaacatgcaaggcatgcctatgagctttttaaaaaggaaggaaatagaccaattatgattgaagaacttgcctcggaacttgggcttagtccatcagtacctgttcatgtagtacttcaggattggataaggcactcagatggaaagcttagtttcttgggatttgtcaggcttctgcatggggtttcttcccgcgcatttcagaaggcttgagagaatgtacacacagttgctattattttttcttgcccgttacctttaaatattttgatggtacctaaatgcatgagctaactccatcagcagacctcagtgggcttctgcaagttgaaggcagtggctgataggaatttgtttctgaagctttatgcaacatatatctatcccacttttccaagatttacaaacacaacctatattccccaccttgtttctcactcactttagtttggtggttctggagttagaagcttgtacagttgaaagctagaccaaaaattgtacccaccttgtcttaagggtttaccccttgtgtacccatggcacctatcaataaacatcacgtgaactccttgataatagacagatatgtagcctttttttcgtagtattttgttgcatttatatgttattattattcaaggccacaagggttggtactttttatataccatagcattattgagttttgaatctataacttcttgcaaactatgcaaatgcaaaacccttttcatcatatatgggtggcatttggttaatgatctatacacaatatctttagatggtaccaaacttctaacgatatcagtgtaaagttttgcttttgaagacattcaaaagcttctggagttcagttttcaatgttagtgattgagtcttaagtttaggatgttaaaaaaaaaaaaaaaaaaa

SEQ ID NO: 2 - protein, 416 aa, Glycine max
MTTAIAIEDVRREVKILRALTGHKNLVQFYEAYEDDDNVYIVMELCKGGELLDRILSRGGKYSEEDARVVMIQILSVVAFCHLQGVVHRDLKPENFLFTSKDDKSTLKAIDFGLSDYVKPDERLNDIVGSAYYVAPEVLHRSYGTEADMWSIGVIAYILLCGSRPFWARTESGIFRAVLKADPSFDEAPWPSLSVDAKDFVKRLLNKDYRKRLTAAQALSHPWLVNHHDDMRIPLDMIIHKLVKAYICSSSLRKSALRALAKTLTVAQLAYLRDQFTLLGPNKSGLISMQNFKTAVLRSSTDASKDSRVLDYVSMVSSIQYRKLDFEEFCAAAISVHQLEGMETWEQHARHAYELFKKEGNRPIMIEELASELGLSPSVPVHVVLQDWIRHSDGKLSFLGFVRLLHGVSSRAFQKA

SEQ ID NO: 3 - DNA, 1520 bp, Glycine max
  CDS (41)...(1201)
caaaggtgcatatgaggatgccgtggctgttcatgttgtgatggaattatgtgcaggtggagagcttttcgataggattattcagcgtggccattataccgagagacaggcagccaagcttactaagactatagttggcgttgtggaagcttgccattctcttggtgtgatgcacagagaccttaaacctgagaattttctctttgtcaatcagcacgaggattcccttctcaaaaccattgactttggattatctgtcttctttaagccaggtgatatatttaatgatgtggtgggcagcccatactatgttgccccggatgttttgcgaaagcgttatggtcctgaggcagatgtttggagtgctggtgttatcctttacattcttttgagtggagtacctccattttgggctgaaaacgaacaaggaatatttgaacaagttctgcgtggtgatcttgacttttcttctgatccctggccttcaatttctgaaagtgcaaaagatttagtaagaaaaatgcttgttcgcgaccctagaaggcggttgactgcacatcaagtattatgtcatccttggattcaagttgatggtgtagctcctgacaagccacttgattctgccgtattaagtcgcttgaagcaattttctgctatgaacaagctcaaaaaaatggcccttataattattgcagagagcttatctgaagaagaaatagctggcttaaaagaaatgttcaagatgatagatgcagataacagtggtcaaatcacttttgaagaacttaaagctggtttgaaaagagtcggcgctaatcttaaggagtctgaaatttatgatttaatgcaagcagctgatgttgataacagtgggacaattgattacggcgagttccttgctgcaacgttgcaccgcaacaaaattgaaagagaagataatctatttgcagccttttcttactttgataaagatggaagtggctatattactcaggaagaacttcaacaggcttgtgatgagtttggcataaaagatgtccgtttggaagagataatcaaggaaattgatgaagataatgatggacgcatagattacaatgagtttgtggctatgatgcagaaaggaaatcttccagcggttggtaagaagggcctagaaaatagcttcagcgttaagttcagggaggcattaaaattgtagttttcattgtcatcattgagttttttttttttttttggtgctctttattaccacacatccctccttttcactttgaaggttcaagattttattcatagtgcaaagtgtttgcatggggatatttaggtgggccttttcatctctgcagttattttgtttgcgtccaatttaaaatggatttttgtaattacgagaatatattaaaaagagatatctttttttattattatattaaaaagagatatctggtggagtattactattagagaggaaatagattatttgaattgaattgacacataaaaaaaaaaaaaaaaaa

SEQ ID NO: 4 - protein, 386 aa, Glycine max
MELCAGGELFDRIIQRGHYTERQAAKLTKTIVGVVEACHSLGVMHRDLKPENFLFVNQHEDSLLKTIDFGLSVFFKPGDIFNDVVGSPYYVAPDVLRKRYGPEADVWSAGVILYILLSGVPPFWAENEQGIFEQVLRGDLDFSSDPWPSISESAKDLVRKMLVRDPRRRLTAHQVLCHPWIQVDGVAPDKPLDSAVLSRLKQFSAMNKLKKMALIIIAESLSEEEIAGLKEMFKMIDADNSGQITFEELKAGLKRVGANLKESEIYDLMQAADVDNSGTIDYGEFLAATLHRNKIEREDNLFAAFSYFDKDGSGYITQEELQQACDEFGIKDVRLEEIIKEIDEDNDGRIDYNEFVAMMQKGNLPAVGKKGLENSFSVKFREALKL

SEQ ID NO: 5 - DNA, 943 bp, Glycine max
ggcttgtaatacgactcactatagggcacgcgtggtcgacggcccgggctggtctgacattctgaggaaagagccagagcatgaaactttcacaagattgagaataactcctcttggtatgaactctgacatgttactcttcctcattctcttgagtgcaaagaaattgctttgaatgggtttttcatgggatgtrtagaatccccctccttcaracacttgtttgattacaatatttgcatatctggaaaatttatagtaaactataattgttatattttttgtttagaggctccgtcacctgatgaaattgaagcttatgtggttctgcaagaatgccttgaaatgagaaaaagatatgtttttagagaagctgttgctccgtgggataaagaagttatatccgaccccagcacacccaagcctaacccagatccatttttatacattcctgaaggaaattctgatgtgagtttttttctcccaccactgaaaacttggatctccctatgttatatgctgtgattaaattagttgtatttctcttgtgctacagcattattttgaaatgcaagatggggttattcgtgtatatccagatagagatggtaagtaacaggaatttgttattgataaacttggtaattttatcaccactcaaggtgggatcttatgcatgcttgcctattacttgcagcaaaagaagagctttttcctgtagctgatgcaactacatttytcacygatcttcatcacttacttcgagtcatagcagcagggaatataagaactttatgccatcataggctcaatcttctagaacaagtacatctctaatttactgaaacaaactgcagcctatgcttgtattttaattacatacaagaatcaattgttgtttgacaatttctgtatttttcagaaattcaatcttcatttgatg

SEQ ID NO: 6 - DNA, 989 bp, Glycine max
  CDS (72)...(746)
gagacccatagtgctgccaaattatagtaactctctttctctatctgaagaagaagaagttctgaagaagcatgtgtggaggtgctatcatctcagacttcattggtgtgaagcgtggccgcaacctcgccgcgcaggaactgtggtccgagcttgaccctttctctgacctccttggcttcgacaccaccaccaccaccaccaccaaccaaccaccccttccagacaaaaaagtggtgtcatcatgtgagaagaagaagaagaaaagtgtgagtgcagaaaagaagagtggtgggcgagctcggaagaacgtgtacagaggaatcaggcaaaggccgtggggcaagtgggccgcggaaataagggacccacataagggcgtccgcgtctggctcggcaccttccccaccgccgaggaagccgcccgagcctacgacgacgccgccaagcgcatccgcggcgacaaggccaagctcaacttcccggccaccgctccgccaccctccaaaaaacaacgctgcctcagccctgacaccaccaccgaacaaagcagcagctcacaatccaccactggatccaccggatcgccgccttccgccgccttccacggcggaggagatgaactcgacctgaaacaacttgaacggtttctagggttggacaacatgggtgctgagtgggacaacatggatgacctgtggatgctggacgacgtcgttgtgcccaaccgtcacttaatttactagaagggagataattaattaattaataaatggcgttttcttaagttatagttttataaaactatgttggtgtatgtgttcttagttttctgttttgtcttgtcctctcgctttggtaatttctgttttgtacggtcgaatgatttcaaaattatgtgcaacgtatcatgagagggatgattatatgttatgattatgattataaataaaggccaattagggtgtgttaaaaaaaaaaaaaaaaaa

SEQ ID NO: 7 - protein, 224 aa, Glycine max
MCGGAIISDFIGVKRGRNLAAQELWSELDPFSDLLGFDTTTTTTTNQPPLPDKKVVSSCEKKKKKSVSAEKKSGGRARKNVYRGIRQRPWGKWAAEIRDPHKGVRVWLGTFPTAEEAARAYDDAAKRIRGDKAKLNFPATAPPPSKKQRCLSPDTTTEQSSSSQSTTGSTGSPPSAAFHGGGDELDLKQLERFLGLDNMGAEWDNMDDLWMLDDVVVPNRHLIY

SEQ ID NO: 8 - DNA, 969 bp, Glycine max
  CDS (49)...(762)
agtaactctctatttctctttccatctctcaagttctgaagaagaaacatgtgtggaggtgctatcatctcagacttcattggtgtgaagcgtggccgcaacctcgccgcgcaggaactatggtctgagcttgaccctttctctgacttccttggcttcgataccaccaattccaaaaaccaaccacccctgcagaaaattccagacaaaaaagtggtgtcatcatgtgagaagaagaagaaaagcgtggtgggtgcagaaaagaagaagagtgatagtgggcgagctcgtaaaaacgtgtacagaggaatcaggcaaaggccatggggcaagtgggccgcggagataagggacccacacaagggtgttcgtgtctggctcggcaccttccccaccgccgaagaagccgcccaagcctacgacgacgccgccatacgcatccgcggcgacaaggccaagctcaacttcccggccaccaccatttccgccgccgccgctccgccaccttccaaaaagcaacgctgcctcagccctgacatcatcactgaagaaagcagcagcagcagctcacattccaccactggatccaccggcgaaagcggcggaggaaacgacgaactcgacctgaaacaaattgaatggtttctaggtttggagaatgagctgcctgttagcaacaacattggtgctgagtgggacaacatggatgacctgtggatgctggacgacgtcgttgtgcccaaccgtcacttaatttactagaaggctaataattaataaatggcgttttcttaagttttagttttactttataaattgtagtataaaactatgttggtatttgtgttcttagttttcttctgctgttttgtctcttctctggctttggtaatttctgttttggacggttgaatgatttcaaaattaggtacttaattacaccctatcaaaaaaaaaaaaaaaaaaaaa

SEQ ID NO: 9 - protein, 237 aa, Glycine max
MCGGAIISDFIGVKRGRNLAAQELWSELDPFSDFLGFDTTNSKNQPPLQKIPDKKVVSSCEKKKKSVVGAEKKKSDSGRARKNVYRGIRQRPWGKWAAEIRDPHKGVRVWLGTFPTAEEAAQAYDDAAIRIRGDKAKLNFPATTISAAAAPPPSKKQRCLSPDIITEESSSSSSHSTTGSTGESGGGNDELDLKQIEWFLGLENELPVSNNIGAEWDNMDDLWMLDDVVVPNRHLIY

SEQ ID NO: 10 - DNA, 856 bp, Glycine max
  CDS (111)...(719)
gtggcggccgctctagaactagtggatcccccgggctgcaggaattcggcacgagcacacattggtgtttccaccaaatacagtgagcaaagttagctgaaaattaaaacatggtttccgccaccgtggattccgattttgcattcttggaatctgttcaacaatacctacttggacatgattccatcaatctcatgtctgaaacccaccaagctgcatctcatgatccattttcagaccctaataaatgtgatggtgattcagggaacattgctttccgaagtgaggatgcaacggctgtggtagcgcgtgatcatgcgccaccaacatggaagcattacagaggggtgaggcgtagaccgtggggaaagtttgcggccgagattagggatccaaagaagaacggagctagggtttggcttggcacgtatgataccgaagagaaggccgctttggcatatgacaaagccgctttcaaaatgcgaggccaaaaggccaagctgaattttcctcatcttattgattccgacaattccgatgaattgtcggagccagtaatgatgacaacttccaagcgaagtttgttagaaatttcatcaccgtcgtcctcgtgttcagatgatagctcagaatcacaagggacaaagaggaggaagagcctggctgaactactgaataaattagccaagaatagaagccaagtcaaggtggaatgttgaagtggctagaggaatatgcatgttgtacaatttgatcaatcattaatatgagacttcaacgattgtaatgtaatctggtgttcatagaattaatgcaattttgttcaccaaaaaaaaaaaaaaaaaaaaaaaaaaaa

SEQ ID NO: 11 - protein, 202 aa, Glycine max
MVSATVDSDFAFLESVQQYLLGHDSINLMSETHQAASHDPFSDPNKCDGDSGNIAFRSEDATAVVARDHAPPTWKHYRGVRRRPWGKFAAEIRDPKKNGARVWLGTYDTEEKAALAYDKAAFKMRGQKAKLNFPHLIDSDNSDELSEPVMMTTSKRSLLEISSPSSSCSDDSSESQGTKRRKSLAELLNKLAKNRSQVKVEC

SEQ ID NO: 12 - DNA, 854 bp, Glycine max
  CDS (115)...(723)
aagctggagctccaccgcggtggcggccgctctagaactagtggatcccccgggctgcaggaattcggcacgagccaccaaatacagtgagcaaagttagctgaaaattaaaacatggtttccgccaccgtggattccgattttgcattcttggaatctgttcaacaatacctacttggacatgattccatcaatctcatgtctgaaacccaccaagctgcatctcatgatccattttcagaccctaataaatgtgatggtgattcagggaacattgctttccgaagtgaggatgcaacggctgtggtggctcgtgatcatgcgccacaaacatggaagcattacagaggggtgagacgtagaccgtggggaaagtttgcggccgagattagggatccaaagaagaacggagctagggtttggcttggcacgtatgataccgaagagaaggcggctttggcatatgacaaagccgctttcaaaatgcgaggccaaaaggccaagctgaattttcctcatcttattgattccgacaattccgatgaattgtcggagccagtaatgatgacaacttccaagcgaagtttgttagaaatttcatcaccgtcgtcctcgtattcagatgatagctcagaatcacaagggacaaagaggaggaagagccttgctgaactactgaataaattagccaagaatagaagccaagtcaaggtggaatgttgaagtggctagatgaatatgcatgttgtacaatttgatcaatcattaatatgagacttcaacgattgtaatgtaatctggtgttcatagaattaatgcaattttgttcaccataaaaaaaaaaaaaaaaaaaa

SEQ ID NO: 13 - protein, 202 aa, Glycine max
MVSATVDSDFAFLESVQQYLLGHDSINLMSETHQAASHDPFSDPNKCDGDSGNIAFRSEDATAVVARDHAPQTWKHYRGVRRRPWGKFAAEIRDPKKNGARVWLGTYDTEEKAALAYDKAAFKMRGQKAKLNFPHLIDSDNSDELSEPVMMTTSKRSLLEISSPSSSYSDDSSESQGTKRRKSLAELLNKLAKNRSQVKVEC

SEQ ID NO: 14 - DNA, 858 bp, Glycine max
  CDS (7)...(648)
aaaacaatgaagaccaaactgctagcatttctcctcttctttgccttgactacaaaaccactactacttggagcagctggagctgctccagagccagtgattgatacatcaggcaagaagctgagagctgatgcaaattaccatatcatccctgcagtgcccttcaccatatgtggctttgttagctgtttcactggtggaggcctttcactagacagcatagatgaatcttgccctcttgatgtaataattgagaaagccaatgaaggcctaccactgagattctcaccagttaacaccaaaaaaggtgttattcgtgtctccaccgatttgaacatttttttctctgattctgatgaaaggtgtccacaccattccactgtgtggatgcttgatcaatttgatgcctctattggacagacatatgtgaccactggtggtgttgttggaaacccgggtgagcacacaattctgaattggttcaagattcagaagtatgaggatgcttataagctggtctattgccctagggtgtgcccctcttgccaccatctgtgcaaggatattggaatgtttgtggatgccaataggagaatgcatctggctctcagtgatgatcccttcaaaattaagttcaaagaagcctgagatcaaagctctttcaaatgatggcaaaattaaatgacaatccatgaatacgtgtgtttataatgatcgatccttgaaattatatttctttgtgaagaattagtaaatgaataaaaaaattaagagtgtatgtttttgtcctgctgttacaactttaatttcactattaaataataaatacaatttttaataaaaaaaaaaaaaaaaaaa

SEQ ID NO: 15 - protein, 213 aa, Glycine max
MKTKLLAFLLFFALTTKPLLLGAAGAAPEPVIDTSGKKLRADANYHIIPAVPFTICGFVSCFTGGGLSLDSIDESCPLDVIIEKANEGLPLRFSPVNTKKGVIRVSTDLNIFFSDSDERCPHHSTVWMLDQFDASIGQTYVTTGGVVGNPGEHTILNWFKIQKYEDAYKLVYCPRVCPSCHHLCKDIGMFVDANRRMHLALSDDPFKIKFKEA

SEQ ID NO: 16 - DNA, 846 bp, Glycine max
  CDS (33)...(662)
cacaaactaattaaccaaaagcatacaaaacaatgaagagcacaatgctgctagcatttgcccttgtcttagccttgagttcacaaccactgctaggaggagctgaagcctcacccgagcaagtggttgacacattaggcaagaagctccgagttggaaccaattactatattgtcccatctcttccctacaccaaaattagaaccactagaggccttggcctagccagtgttggaaaaccttattgtcctcttgatgttgtggttgtgaatggataccatggcttgccagtgacattctcaccagttaatcctaagaaaggggtcattcgtgtctcaactgatttgaacatcaagttctctgctcgcactagttgtccccgccaatattccacggtttggaaacttgatgattttgatttctcaaagagacaatggtttgtgaccactggtggtgttgtgggaaaccctagcttggaaaccatccacaactggttcaagattgagaagtacgatggtgcttacaaattggtctattgtcccagcgtggtgaaatgtccaaagcatttgtgcaagaatgttgggttgtttgtggatgagaaagggaacaagcgtcttgctctcactgatgttcccctcaaagttcaattccaacaagcctaaacaagcttaatgctcctctaagtctaacattaatgcataaaaactatatatgaataagtgtatttcctttctaataacgatgcatgttctttcaatgtttatcaggatcctcatgtaaggttttccttggtaaatgcaaaataaataaaatgaacaaatgatataaaaaaaaaaaaaaaaaaaa

SEQ ID NO: 17 - protein, 209 aa, Glycine max
MKSTMLLAFALVLALSSQPLLGGAEASPEQVVDTLGKKLRVGTNYYIVPSLPYTKIRTTRGLGLASVGKPYCPLDVVVVNGYHGLPVTFSPVNPKKGVIRVSTDLNIKFSARTSCPRQYSTVWKLDDFDFSKRQWFVTTGGVVGNPSLETIHNWFKIEKYDGAYKLVYCPSVVKCPKHLCKNVGLFVDEKGNKRLALTDVPLKVQFQQA

SEQ ID NO: 18 - DNA, 1410 bp, Glycine max
  CDS (31)...(1095)
cggcacgagaagcaaacataacttgcagtaatggcttcaatgaataaccaaaaagaaattgagctctttgagggccaatctcttctgtacatgcagctatatgggcacctaagacctatgtgtcttaagtgggctgttcaactaggtattccagacataatacagaaccatgccaaacccatttctctttctgacttggtctctactcttcaaattccaccagctaacgctgcttttgtgcagcggttcatgcgcttcttggcacacaatggaatctttgagatccatgagagccaagaagatcatgaactaacatatgctctaacccctgcatcaaagcttcttgtcaatagtagtgatcattgtctatctccaatggttctagcgtttaccgatccacttcggaacgttaaataccatcacttgggggaatggattcgtggggaggacccctcagtatttgagacagcccacggaacaagcgcttggggacttcttgagaaaaatcctgaatattttagtctcttcaatgaggctatggcaagtgattcccgaatagtagacttggcactcaaaaattgcacttcagtttttgaggggctagattccatggtggatgttggtggtggaactggaaccacggccagaattatctgtgacgcatttcctaagttgaaatgtgttgtgcttgaccttcctcatgttgtagagaacttgacagggaccaataatttgagttttgttggtggtgacatgttcaactctatccctcaagctgatgcagtgctactaaagtgggttttacataattggaccgacgaaaattgcataaagatcctgcaaaagtgtagagattctatttcaagcaaaggcaacagtggaaaagtgattatcatagatgccgtaataaatgagaagctagatgacccggatatgacacaaacaaagcttagtttggacattattatgttgacgatgaatggaagagagagaacggaaaaagaatggaaacaactcttcatcgaagcaggattcaaacactacaaaatatttcccatctttggttttagatctctgattgaggtctatccttgaacatttttatgatgtgtatgtcacacttaacgtttatatttatgaacatcctcagacatcgttgtaattgtatttagtggtttgcgtgttgtttgctgaataaagctatgatgacatagcattatcaacttctggtggaattacattatgttcctccccgctatgttgttaaatgttctgtgtacggatttctcattcctatttggccaaaataaattgaataaacatgtaatggatgcaatcaatatacaatgttttggtatggctttacgtctttaaaaaaaaaaaaaaaaaaaaaaaaaaaaaaaaaaaaaa

SEQ ID NO: 19 - protein, 354 aa, Glycine max
MASMNNQKEIELFEGQSLLYMQLYGHLRPMCLKWAVQLGIPDIIQNHAKPISLSDLVSTLQIPPANAAFVQRFMRFLAHNGIFEIHESQEDHELTYALTPASKLLVNSSDHCLSPMVLAFTDPLRNVKYHHLGEWIRGEDPSVFETAHGTSAWGLLEKNPEYFSLFNEAMASDSRIVDLALKNCTSVFEGLDSMVDVGGGTGTTARIICDAFPKLKCVVLDLPHVVENLTGTNNLSFVGGDMFNSIPQADAVLLKWVLHNWTDENCIKILQKCRDSISSKGNSGKVIIIDAVINEKLDDPDMTQTKLSLDIIMLTMNGRERTEKEWKQLFIEAGFKHYKIFPIFGFRSLIEVYP

SEQ ID NO: 20 - DNA, 1630 bp, Glycine max
  CDS (93)...(1403)
cacctatagtactctcaccttctctcaatcatttttcattcatttgtcaacttttatagacctcgattagtgtatgcaaatttctttaggacatgtggcctacattagtggctaataagatattcaagaagagacttggaagcagcaattttatagcggattatcctagttataaagaacccttgctgggtattgtagacattgatcagaactcaaaaaccattcttaatgatcacaaggactcacacaaatacaaggtatttgttagtacatggaacgtaggtgggattgcaccggatgaagatttgaatatagatgatttgttggagacatgcaacaactcttgtgacatctatatactagggtttcaagaaatagtgcctctaaaagcatcaaatgtattggggtccgaaaacaatgagatttctatgaaatggaattccataatcagggaagccttgaacaagaaaataacacatcaaagggacaaagatgctaaaaaacaggagctaaagaataattttcccaataagaaagaaaatccagctaagtgctgtgatgccccacatgatttccaatgtatcattagcaagcaaatggttggattattcatatctgtgtggattagaagagatctttgtccattcattcggcatccaagcgtgtcatgtgtaggttgtgggataatgggctgcttaggaaacaagggttctatatcagtgagatttcagttacatgaaaccagcttctgctttgtgtgcagccatctagcttcagggggcagagaaggggatgagaagcacaggaactctaatgttgctgaaattttttctcggacaagttttcctagaggccctttgcttgatttgcctagaaccattcttgatcatgatcatgtaatattgcttggagatctaaattacagaatttctctaccagaagaaaccacacgcttgcttgttgaaaaaagagactgggattctttattagcaaatgatcagctaataatggagctaatgagtggaaacatgttaagaggatggcacgaaggagcaattaaatttgcacctacctacaaatattgtccaaattcagacatttactatggatgctgctatcatggcaaaaaggcagaaaagagaagagcaccagcatggtgtgatcgaatagtatggtgcggtgagggtctaaagcaacttcagtacactagaattgaatcaaaactatcagatcataggcctgttaaggcaatgtttatagcagaagtcagggttttaccagagctgatgaaaaacttgcaaagcttgttcctatcagaaagatacgagcaaattaaaactccctttgaagtttccaccactgaagattttgtaaatagaaaacgatcaagcttccggttgtgaattttttgtgtgattcaagctaggctttaaattgtgattgtgattaatgttgcaattttatcacaatcttttgtggagaaaaatttgcataaaaatgtgattgatgtgaccgttattacaatcagggacttcaacaaccgtgacattgtcatcactgttgtggttgcacatggtttttttctataaaaaaaaaactgtgatatatgtaaaaaaaaaaaaaaaaaaaa

SEQ ID NO: 21 - protein, 436 aa, Glycine max
MWPTLVANKIFKKRLGSSNFIADYPSYKEPLLGIVDIDQNSKTILNDHKDSHKYKVFVSTWNVGGIAPDEDLNIDDLLETCNNSCDIYILGFQEIVPLKASNVLGSENNEISMKWNSIIREALNKKITHQRDKDAKKQELKNNFPNKKENPAKCCDAPHDFQCIISKQMVGLFISVWIRRDLCPFIRHPSVSCVGCGIMGCLGNKGSISVRFQLHETSFCFVCSHLASGGREGDEKHRNSNVAEIFSRTSFPRGPLLDLPRTILDHDHVILLGDLNYRISLPEETTRLLVEKRDWDSLLANDQLIMELMSGNMLRGWHEGAIKFAPTYKYCPNSDIYYGCCYHGKKAEKRRAPAWCDRIVWCGEGLKQLQYTRIESKLSDHRPVKAMFIAEVRVLPELMKNLQSLFLSERYEQIKTPFEVSTTEDFVNRKRSSFRL

SEQ ID NO: 22 - DNA, 1600 bp, Glycine max
agcaccatcatctctatcattcggaatgcaaccaagctaaaagattactacaactaattgcttttccttatcacattttctgacgtatagtgtgattttatatatttttacataagagaaataatttctacaaaaaaaatgttcgataaaataagtagagaacgtgtattaataatttctacataagaaataaagaaatatattagatataataagtgatgcaagaaagatggacaaaaataattacgtaaatatcattctataaatttattattcattatataaatagcattaccattgttgaaacttgaaagtgggtccatcgtttacaactaaagaaagacaccctagcgtaaaatattcaaccatcgacgtctacttcaattaacatgaagatgtagttccatctcaacggatttccgtctcaaataaaattcttaataacgtgctactaaccattggaatctgcagaatatctcgtttagttgggcacaatccctcaaaagcgatgtatttttttaatggaaacaatgcatgccacaagaacgtttatatatacataattttactaaacaaatcgtaatacaaaactttattattataacgtgatttgtcactttttgcttcagaaaaatactttgtacaaaacattaagacaataacataagttgccaataccatacataaaactctttaatgaatcataatgatgaaaattgagagatatttagttccatgataaagagtgtgtttgtgtgggaatttgaccaaacgcaattgttgttccagtgaaaacttttctcgcgtgtttggccttttgtgtctcagaaagctaattttctccatttaacgtggtttggacccattttcaaacgcactcacagtgagtccgtttctgtagaagtccttccagcaggccacccctcccccaaagaaaaatttaagaaacaagaagagaaagaaaaaaagaaaaagaaaaaacaagtcacatattttattcttatgtcagccaaaaacttgactagctgtagatggggcaataataactagctattcatcacatttcctagctaattgcctgttttgttatggaccacattcccacttgcactcatcttcagcaatttaaattaggtaataaacattaagatatcctttaaaatctactcaacaaacagaagaattcaaatctgcaagaagggtagacccatgttttatactagctttctctatctctctcccactgggacataaatgttcctatatttcaaaaaatatatatatgatatgatgagcaatgcagccaaaggtgcatcatcttttacgtcacatgaaagcctttcctacctcttcaagctgcacaagcctttctctttcccagaatgatttttttccatttcttgttattattactccttttggactttctatataatgctttctatatacgtttccaataataccacgtacactactcatgtgccaggaaaaggagcagcagtgaccacctagcaatagtactctcgccttctctcaatcatttttcatttgtcaacttttatagacctcgatttgtgtatgcaaatttctttaggacatg

SEQ ID NO: 23 - protein, 465 aa, Zea mays
MEDVRATYSMGKELGRGQFGVTHLCTHRTSGEKLACKTIAKRKLAAREDVDDVRREVQIMHHLSGQPNVVGLRGAYEDKQSVHLVMELCAGGELFDRIIARGQYTERGAAELLRAIVQIVHTCHSMGVMHRDIKPENFLLLSKDEDAPLKATDFGLSVFFKEGELLRDIVGSAYYIAPEVLKRKYGPEADIWSVGVMLYIFLAGVPPFWAENENGIFTAILRGQLDLSSEPWPHISPGAKDLVKKMLNINPKERLTAFQVLNHPWIKEDGDAPDTPLDNVVLDRLKQFRAMNQFKKAALRIIAGCLSEEEITGLKEMFKNIDKDNSGTITLDELKHGLAKHGPKLSDSEMEKLMEAADADGNGLIDYDEFVTATVHMNKLDREEHLYTAFQYFDKDNSGYITKEELEHALKEQGLYDADKIKDIISDADSDNDGRIDYSEFVAMMRKGTAGAEPMNIKKRRDIVL

SEQ ID NO: 24 - protein, 490 aa, Arabidopsis thaliana
MANKPRTRWVLPYKTKNVEDNYFLGQVLGQGQFGTTFLCTHKQTGQKLACKSIPKRKLLCQEDYDDVLREIQIMHHLSEYPNVVRIESAYEDTKNVHLVMELCEGGELFDRIVKRGHYSEREAAKLIKTIVGVVEACHSLGVVHRDLKPENFLFSSSDEDASLKSTDFGLSVFCTPGEAFSELVGSAYYVAPEVLHKHYGPECDVWSAGVILYILLCGFPPFWAESEIGIFRKILQGKLEFEINPWPSISESAKDLIKKMLESNPKKRLTAHQVLCHPWIVDDKVAPDKPLDCAVVSRLKKFSAMNKLKKMALRVIAERLSEEEIGGLKELFKMIDTDKSGTITFEELKDSMRRVGSELMESEIQELLRAADVDESGTIDYGEFLAATIHLNKLEREENLVAAFSFFDKDASGYITIEELQQAWKEFGINDSNLDEMIKDIDQDNDGQIDYGEFVAMMRKGNGTGGGIGRRTMRNSLNFGTTLPDESMNV

SEQ ID NO: 25 - protein, 205 aa, Lycopersicon esculentum
MKINQLFFPFLILAISFNSLLSSAAESPPEVVDIDGKILRTGVDYYILPVVRGRGGGLTMDSIGDKMCPLDAVVQEHNEIDQGLPLTFTPVDPKKGVIRESTDLNIIFSANSICVQTTQWKLDDFDETTGQYFITLGGDQGNPGVETISNWFKIEKYDRDYKLLYCPTVCDFCKVICRDIGIFIQDGVRRLALSDVPFKVMFKKA

SEQ ID NO: 26 - protein, 210 aa, Nicotiana tabacum
MKTNQLFLPFLIFTISFNSFLSSSAEAPPAVVDIAGKKLRTGIDYYILPVVRGRGGGLTLDSTGNESCPLDAVVQEQQEIKNGLPLTFTPVNPKKGVIRESTDLNIKFSAASICVQTTLWKLDDFDETTGKYFITIGGNEGNPGRETISNWFKIEKFERDYKLVYCPTVCNFCKVICKDVGIFIQDGIRRLALSDVPFKVMFKKAQVVKD

SEQ ID NO: 27 - protein, 364 aa, Zea mays
MELSPNNSTDQSLLDAQLELWHTTFAFMKSMALKSAIHLRIADAIHLHGGAASLSQILSKVHLHPSRVSSLRRLMRVLTTTNVFGTQPLGGGSDDDSEPVYTLTPVSRLLIGSQSSQLAQTPLAAMVLDPTIVSPFSELGAWFQHELPDPCIFKHTHGRGIWELTKDDATFDALVNDGLASDSQLIVDVAIKQSAEVFQGISSLVDVGGGIGAAAQAISKAFPHVKCSVLDLAHVVAKAPTHTDVQFIAGDMFESIPPADAVLLKSVLHDWDHDDCVKILKNCKKAIPPREAGGKVIIINMVVGAGPSDMKHKEMQAIFDVYIMFINGMERDEQEWSKIFSEAGYSDYRIIPVLGVRSIIEVYP

SEQ ID NO: 28 - protein, 352 aa, Medicago sativa
MASSINGRKPSEIFKAQALLYKHIYAFIDSMSLKWAVEMNIPNIIQNHGKPISLSNLVSILQVPSSKIGNVRRLMRYLAHNGFFEIITKEEESYALTVASELLVRGSDLCLAPMVECVLDPTLSGSYHELKKWIYEEDLTLFGVTLGSGFWDFLDKNPEYNTSFNDAMASDSKLINLALRDCDFVFDGLESIVDVGGGTGTTAKIICETFPKLKCIVFDRPQVVENLSGSNNLTYVGGDMFTSIPNADAVLLKYILHNWTDKDCLRILKKCKEAVTNDGKRGKVTIIDMVIDEKKDENQVTQIKLLMDVNMACLNGKERNEEEWKKLFIEAGFQHYKISPLTGFLSLIEIYP

SEQ ID NO: 29 - protein, 664 aa, Arabidopsis thaliana
MSPVEPAGIMKKSHRQKSQRLWAKLVMRKWLNISGRDPEYGADTDNESENEDAREDNDDSSSDEEGGSGSRGRESKVYENAEDAIAAASAVVDAAAAAAEFISNDAPMKLRRRNSETLRAQYINNKEIRVCVGTWNVGGISPPSDLDIDDWIEINQPADIYVLGSQEIVPLNAGNILGAEDDRPVAKWEEVIREALNRVRPKLSGVKSYSDPPSPGRFKPFEETHDIIEEEVAFESDSDAGVEIHPIDEEEEEETDRLWALKHDGGVIGEVKTLVDPNTGLPVVEIKRQFSIPKKLDRQLCLRADSFKGISDDDSTQTGMKTINRMLSGKERIGLSWPEPPLNMLGPCVLDRQPSIKTVKSLKTAKSFKAYSSFKSVAGNNNGIPPEVLALAEMDLKLLMERKRRPAYVRLVSKQMVGILLTIWVKRSLRKHIQNVRVSTVGVGVMGYIGNKGAVSVSMSINQTFFCFINTHLTAGEREVDQIKRNADVHEIHKRTVFHSVSALGLPKLIYDHERIIWLGDLNYRLSSSYEKTRDLISKREWSKLLEYDQLVKEYRKGRAFDGWSEGTLHFPPTYKYQANSDEYTANDGKAPKRTPAWCDRVLSYGKGMRLVHYRRTEQKFSDHRPVTAIYMAEVEVFSARKLQRALTFTDAEIEDEGLVAVLV

SEQ ID NO: 30 - protein, 4 aa, Artificial Sequence
  cAMP and cGMP-dependent protein phosphorylation site
RKRS

SEQ ID NO: 31 - protein, 246 aa, Arabidopsis thaliana
MCGGAIISDYAPLVTKAKGRKLTAEELWSELDASAADDFWGFYSTSKLHPTNQVNVKEEEAVKKEQATEPGKRRKRKNVYRGIRKRPWGKWAAEIRDPRKGVRVWLGTFNTAEEAAMAYDVAAKQIRGEKAKLNFPDLDHHPSTPPPSSTSLRLSDQPPAKKVCVVSQSELAQPSFPVECVGFGKGEEFQNLMYGFEPDYDLKQQISSLESFLELDGTTAEQPSQLDESVCDVDMWMLDDVIASYE

SEQ ID NO: 32 - protein, 248 aa, Arabidopsis thaliana
MCGGAIISDYAPLVTKAKGRKLTAEELWSELDASAADDFWGFYSTSKLHPTNQVNVKEEAVKKEQATEPGKRRKRKNVYRGIRKRPWGKWAAEIRDPRKGVRVWLGTFNTAEEAAMAYDVAAKQIRGDKAKLNFPDLHHPPPPNYTPPPSSPRSTDQPPAKKVCVVSQSESELSQPSFPVECIGFGNGDEFQNLSYGFEPDYDLKQQISSLESFLELDGNTAEQPSQLDESVSEVDMWMLDDVIASYE

SEQ ID NO: 33 - DNA, 1908 bp, Glycine max
  CDS (24)...(1454)
gcacgagattcaatcttcatttgatgctaaatgcggatagagaatttcttgctcagaagagtgctccacatcgagacttctataatgttagaaaagttgatactcatgtccaccactcagcatgcatgaatcagaaacatcttttaaggttcataaagtcaaagctgagaaaagagcctgatgaggttgtaatatttcgagatgggacatatctaacgttggaagaggttttcaagagtttagatttgtctggatatgacctcaatgttgaccttttggacgttcacgcagacaagagtacttttcatcgctttgataagttcaatcttaaatacaatccttgcggtcaaagtaggctcagggagatatttcttaagcaggataatctcattcaaggtcgttttcttggtgagttaactaagcaagtgttttcagatcttgctgccagtaaatatcagatggctgaatatagaatatcaatatatggtaggaagcaaagtgagtgggaccaactagccagttggatagtgaataatgatttgtacagcgagaatgtcgtttggttgattcagcttccacggttgtacaatgtgtacaaagaaatgggaattgtgacatcattccagaacatgctcgacaatattttcattccactttttgaggtcactgtcaacccagattcacatcctcagctgcatgttttcctgaaacaggttgttgggttggatttggttgatgatgaaagcaaacctgaaagacggccaacaaaacacatgcctacacctgagcaatggactaatgttttcaatccggcattttcatactatgtctattactgttatgcaaatctttacaccttaaacaagcttcgagaatcaaagggaatgacaacaatcaaattccgtccacattctggagaggctggtgatattgaccaccttgcagcaacctttctcacggctcacaacattgcacatggaatcaatttgaaaaaatctcctgtgcttcaatatttatattatttagcccagattgggctggcaatgtctcctttgagcaataactccctattcttagactaccatcggaatccttttccaatgttcttcttacggggtctgaatgtgtcactttctactgatgatcctctccaaattcacttaacaaaggaaccattggttgaagaatatagcatagctgcttctgtgtggaagttgagctcatgtgatttatgtgagattgcccgtaattcagtttatcaatcaggtttctcacatgctttaaagtcacattggattggtaaggagtactacaagagtgggccacgcggaaatgacattcagagaacaaacgttcctcacatccggttggaattccgtgatacgatttggagagaggagatgcaacaggtttatttgggcaaagccatcattcctgaagtagtagacaaataagatgccagggggcattcctttcatacacatgaggtagatagtatcctcaaacccttgcagcgtcagagatgcagggctgaagacatacaatgctggtctgatgatatgagccaggtacatactatagagaatcaacaagatcgatgagcttgagcatcgaacaaggttttacagctacaaatcgacgtacccgtggggttttttctatccttgggttagagttaaaattattagggttagcaattagttcatgcttcaagcaaaatgatgcctgtcggtgttagaaatgtatgttagtaatgtaagttatgatcctttgaagggagagagatattcatctcccaaaattttgattcccattgtttatcgataataatatatcgtgaacgacagttgagacttgaaaaacggtggctctattagttttaccgtaaaaaaaaaaaaaaaaaaaaaa

SEQ ID NO: 34 - protein, 476 aa, Glycine max
MLNADREFLAQKSAPHRDFYNVRKVDTHVHHSACMNQKHLLRFIKSKLRKEPDEVVIFRDGTYLTLEEVFKSLDLSGYDLNVDLLDVHADKSTFHRFDKFNLKYNPCGQSRLREIFLKQDNLIQGRFLGELTKQVFSDLAASKYQMAEYRISIYGRKQSEWDQLASWIVNNDLYSENVVWLIQLPRLYNVYKEMGIVTSFQNMLDNIFIPLFEVTVNPDSHPQLHVFLKQVVGLDLVDDESKPERRPTKHMPTPEQWTNVFNPAFSYYVYYCYANLYTLNKLRESKGMTTIKFRPHSGEAGDIDHLAATFLTAHNIAHGINLKKSPVLQYLYYLAQIGLAMSPLSNNSLFLDYHRNPFPMFFLRGLNVSLSTDDPLQIHLTKEPLVEEYSIAASVWKLSSCDLCEIARNSVYQSGFSHALKSHWIGKEYYKSGPRGNDIQRTNVPHIRLEFRDTIWREEMQQVYLGKAIIPEVVDK

(19) United States
(12) Patent Application Publication
(10) Pub. No.: US 2005/0070697 A1
(43) Pub. Date: Mar. 31, 2005

(54) Pathogen-responsive genes, promoters, regulatory elements, and methods of use for same

(75) Inventors: Xu Hu, Urbandale, IA (US); Guihua Lu, Johnston, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/675,086

(22) Filed: Sep. 30, 2003

Related U.S. Application Data
(60) Provisional application No. 60/414,771, filed on Sep. 30, 2002.

(51) Int. Cl. C07H 21/02; C07H 21/04

(52) U.S. Cl. 536/23.1

(57) ABSTRACT

The present invention relates to nematode-regulated polypeptides, nucleotide sequences encoding the same and regulatory elements and their use in creating or enhancing pathogen resistance in plants. Nucleic acid constructs comprising a nematode-control sequence operably linked to a promoter, or other nucleotide sequence operably linked to a nematode specific regulatory region are disclosed as well as vectors, plant cells, plants, and transformed seeds containing such constructs are provided. Methods for the use of such constructs in repressing or inducing expression of a nematode-control sequences in a plant are also provided. In addition, methods are provided for conferring or improving pathogen resistance in plants by repression or induction of nematode-control sequences or by spatially and temporally directing expression to pathogen invasion.

### CROSS-REFERENCE TO RELATED APPLICATIONS

This application claims the benefit of U.S. Provisional Application No. 60/414,771, filed Sep. 30, 2002.

### FIELD OF THE INVENTION

This invention relates to compositions and methods useful in creating or enhancing pathogen-resistance in plants. Additionally, the invention relates to plants and other organisms which have been genetically transformed with the compositions of the invention.

### BACKGROUND OF THE INVENTION

Plants are continually attacked by a diverse range of phytopathogenic organisms. These organisms cause substantial losses to crops each year. Traditional approaches for control of plant diseases have been the use of chemical treatment and the construction of interspecific hybrids between resistant crops and their wild-type relatives as sources of resistant germplasm. However, environmental and economic concerns make chemical pesticides undesirable, while traditional interspecific breeding is inefficient and often cannot eliminate the undesired traits of the wild species. Thus, the discovery of pest and pathogen-resistant genes provides a new approach to control plant diseases.

Pathogen infection, such as Nematode infection, is a significant problem in the farming of many agriculturally significant crops. For example, soybean cyst nematode (Heterodera glycines, herein referred to as “SCN”) is a widespread pest that causes substantial damage to soybeans every year. Such damage is the result of the stunting of the soybean plant caused by the cyst nematode. The stunted plants have smaller root systems, show symptoms of mineral deficiencies in their leaves, and wilt easily. The soybean cyst nematode is believed to be responsible for yield losses in soybeans that are estimated to be in excess of $1 billion per year in North America. Other pathogenic nematodes of significance to agriculture include the potato cyst nematodes Globodera rostochiensis and Globodera pallida, which are key pests of the potato, while the beet cyst nematode Heterodera schachtii is a major problem for sugar beet growers in Europe and the United States.

The primary method of controlling nematodes has been through the application of highly toxic chemical compounds. The widespread use of chemical compounds poses many problems with regard to the environment because of the non-selectivity of the compounds and the development of insect resistance to the chemicals. Nematicides such as Aldicarb and its breakdown products are known to be highly toxic to mammals.

As a result, government restrictions have been imposed on the use of these chemicals.

The most widely used nematicide, methyl bromide, is scheduled to be soon retired from use, and at present, there is no promising candidate to replace this treatment. Thus, there is a great need for effective, non-chemical methods and compositions for nematode control.

Various approaches to pest control have been tried including the use of biological organisms which are typically “natural predators” of the species sought to be controlled. Such predators may include other insects, fungi, and bacteria such as Bacillus thuringiensis. Alternatively, large colonies of insect pests have been raised in captivity, sterilized and released into the environment in the hope that mating between the sterilized insects and fecund wild insects will decrease the insect population. While these approaches have had some success, they entail considerable expense and present several major difficulties. For example, it is difficult both to apply biological organisms to large areas and to cause such living organisms to remain in the treated area or on the treated plant species for an extended time. Predator insects can migrate and fungi or bacteria can be washed off of a plant or removed from a treated area by rain. Consequently, while the use of such biological controls has desirable characteristics and has met with some success, in practice these methods have not achieved the goal of controlling nematode damage to crops.

Advances in biotechnology in the last two decades have presented new opportunities for pest control through genetic engineering. In particular, advances in plant genetics coupled with the identification of insect growth factors and naturally-occurring plant defensive compounds or agents offer the opportunity to create transgenic crop plants capable of producing such defensive agents and thereby protect the plants against insect attack and resulting plant disease.

Transgenic plants that are resistant to specific insect pests have been produced using genes encoding Bacillus thuringiensis (Bt) endotoxins or plant protease inhibitors (PIs). Transgenic plants containing Bt endotoxin genes have been shown to be effective for control of some insects (see Atkinson et al., (2003) Annu. Rev. Phytopathol. 41:26.1-26.25). Effective plant protection using transgenically inserted PI genetic material has not yet been demonstrated in the field. While cultivars expressing Bt genes may presently exhibit resistance to some insect pests, resistance based on the expression of a single gene might eventually be lost due to the evolution of Bt resistance in the insects. Thus, the search for additional genes which can be inserted into plants to provide protection from insect pests is needed.

Additional obstacles to pest control are posed by certain pests. For example, it is known that certain nematodes, such as the soybean cyst nematode (“SCN”), can inhibit certain plant gene expression at the nematode feeding site (see Gheysen and Fenoll (2002) Annu Rev Phytopathol 40:191-219). Thus, in implementing a transgenic approach to pest control, an important factor is to increase the expression of desirable genes in response to pathogen attack. Consequently, there is a continued need for the controlled expression of genes deleterious to pests in response to plant damage.

One promising method for nematode control is the production of transgenic plants that are resistant to nematode infection and reproduction. For example, with the use of nematode-inducible promoters, plants can be genetically altered to express nematicidal proteins in response to exposure to nematodes. See, for example, U.S. Pat. No. 6,252,138, herein incorporated by reference. Alternatively, some methods use a combination of both nematode-inducible and nematode-repressible promoters to obtain nematode resistance. Thus, WO 92/21757, herein incorporated by reference, discusses the use of a two promoter system for disrupting nematode feeding sites where one nematode-inducible promoter drives expression of a toxic product that kills the plant cells at the feeding site while the other nematode-repressible promoter drives expression of a gene product that inactivates the toxic product of the first promoter under circumstances in which nematodes are not present, thereby allowing for tighter control of the deleterious effects of the toxic product on plant tissue. Similarly, with the use of proteins having a deleterious effect on nematodes, plants can be genetically altered to express such deleterious proteins in response to nematode attack.

Although these methods have potential for the treatment of nematode infection and reproduction, their effectiveness is heavily dependent upon the characteristics of the nematode-inducible or nematode-repressible promoters discussed above, as well as the deleterious properties of the proteins thereby expressed. Thus, such factors as the strength of such nematode-responsive promoters, degree of induction or repression, tissue specificity, or the like can all alter the effectiveness of these disease resistance methods. Similarly, the degree of toxicity of a gene product to nematodes, the protein's longevity after consumption by the nematode, or the like can alter the degree to which the protein is useful in controlling nematodes. Consequently, there is a continued need for the identification of nematode-responsive promoters and nematode-control genes for use in promoting nematode resistance.

### SUMMARY OF THE INVENTION

Compositions and methods involved in plant defense signaling pathways for promoting nematode and other pest resistance in plants are provided. The compositions include nucleic acid molecules comprising a sequence useful in pathogen control as well as pathogen induced regulatory elements. The invention further includes expression constructs comprising nucleic acid sequences, operably linked to regulatory promoters, including the pathogen regulatory promoter elements of the invention, the nucleic acid sequences encoding proteins useful in pathogen control of the invention or other combinations of these novel sequences of the invention with other nucleotide sequences, as well as vectors and transformed plant cells, plants and seeds comprising these constructs. The pathogen control sequences include novel proteins which are either up or down regulated in response to pathogen infection and are involved in formation of cyst nematode or other nematode syncyntia. These proteins, the nucleotide sequences encoding them and the regulatory elements associated with them provide an opportunity to manipulate defense signaling pathways in plants to engineer plants with improved resistance to plant pathogens. The proteins of the invention include calcium dependent protein kinases (hereinafter CDPK), AP2-like proteins (named nematode-responsive transcription factor 1, hereinafter NRTF1), inositol 5-phosphatases (hereinafter IPP), caffeic acid 7-O-methyltransferases (hereinafter 7OM), adenosine-5′-phosphate deaminase (AMPD), and nematode-responsive proteins (hereinafter NRP). Amino acid sequences of these proteins are provided as well as purified proteins themselves. Polynucleotides having nucleic acid sequences encoding CDPK, NRTF1, IPP, 7OM, AMPD and NRP polypeptides are also provided. The DNA sequences encoding these proteins can be used to transform plants, bacteria, fungi, yeasts, and other organisms for the control of pests.

In yet another embodiment, regulatory regions capable of conferring spatial and temporal expression that is pathogen invasion specific are provided. These comprise promoters that are natively associated with the nucleotide sequences encoding the proteins of the invention as well as their functional equivalents. In addition to these promoter sequences, the nematode-regulated promoters of the invention encompass fragments and variants of these particular promoters as defined herein. Nucleotide sequences of promoter regulatory elements of two of such proteins are provided. Further the nucleotide sequences encoding the proteins disclosed herein can be used to isolate promoters of the genes of the invention using standard molecular protocols as described and incorporated by reference herein. These promoter elements can also be used to isolate other signaling components associated with regulation of these genes in response to pathogen invasion, and can be used to engineer synthetic pathogen-regulatory promoters.

The polynucleotides of the invention, or at least 20 contiguous bases therefrom, may be used as probes to isolate and identify similar genes in other plant species.

In one aspect, this invention relates to DNA sequences isolated from soybean (Glycine max). These sequences alone, or in combination with other sequences, can be used to improve the nematode, or other pathogen, resistance which involves formation of syncytia in a plant. In another aspect of the present invention, expression cassettes and transformation vectors comprising the isolated nucleotide sequences are disclosed. The transformation vectors can be used to transform plants and express the pathogen control genes in the transformed cells. In this manner, the pathogen resistance, particularly nematode resistance, of plants can be improved. Transformed cells as well as regenerated transgenic plants and seeds containing and expressing the isolated DNA sequences and protein products are also provided.

### BRIEF DESCRIPTION OF THE DRAWINGS

FIG. 1 shows an amino acid sequence alignment of soybean CDPKa (SEQ ID NO: 2), CDPKb (SEQ ID NO: 4), maize CDPK (L27484, SEQ ID NO: 23), and Arabidopsis CDPK (U20388, SEQ ID NO: 24).

FIG. 2 shows the 5′-flanking region of the adenosine-5′-phosphate deaminase (AMPD) gene (SEQ ID NO: 5). The first MET codon and potential TATA box are bolded.

FIG. 3 shows an amino acid alignment of soybean NRTF1a (SEQ ID NO: 7), NRTF1b (SEQ ID NO: 9), with two Arabidopsis thaliana AP2 proteins, AJ001911 (SEQ ID NO: 31) and AF003096 (SEQ ID NO: 32), with the conserved AP2-domain indicated by underlining.

FIG. 4 shows the amino acid sequence alignment of soybean NRTF1a-NRTF1d (SEQ ID NOs: 7, 9, 11, and 13), with the conserved AP2-domain indicated by underlining.

FIG. 5 shows the amino acid sequence alignment of soybean NRP-1 (SEQ ID NO: 15), NRP-2 (SEQ ID NO: 17), tomato miraculin homologue (T07871, SEQ ID NO: 25), and tobacco tumor-related protein (T03803, SEQ ID NO: 26).

FIG. 6 shows the amino acid sequence alignment of soybean, maize and Medicago 7OM proteins. The soybean 7OM protein (SEQ ID NO: 19) has 51% similarity and 40% identity to a maize 7OM homologue (L14063, SEQ ID NO: 27), and 68% similarity and 58% identity to a Medicago 7OM homologue (AF000975, SEQ ID NO: 28).

FIG. 7 shows the amino acid sequence alignment of soybean IPP (SEQ ID NO: 21) and Arabidopsis IPP (AY048296, SEQ ID NO: 29). There is 57% similarity and 46% identity between the soybean and Arabidopsis IPP encoded proteins. The potential CAMP and cGMP-dependent protein phosphorylation site is underlined.

FIG. 8 shows the 5′-flanking region of IPP gene (SEQ ID NO: 22). The first MET codon and potential TATA box are bolded.

### DETAILED DESCRIPTION OF THE INVENTION

The present invention provides, inter alia, compositions and methods for promoting pathogen resistance in plants, more particularly for improving nematode resistance of plants. The compositions of the invention are nucleic acid molecules comprising sequences useful in improving nematode resistance in plants. These compositions can be transferred into plants to confer or improve nematode resistance in the transformed plants. By “confer or improve nematode or other such pathogen resistance” is intended that the proteins, DNA, or RNA sequences, either alone or in combination with other proteins or sequences, enhance resistance of a plant to nematodes and nematode-caused damage or to other pathogens which cause a similar plant reaction. In this manner, resistance to nematodes and other such pathogens can be enhanced or improved in the transformed plant when at least one of the sequences of the invention is provided.

The compositions comprise nucleic acid molecules comprising sequences of plant genes and the polypeptides encoded thereby. Particularly, the nucleotide and amino acid sequences for two soybean CDPKs, four AP2 like proteins (named NRTF1a, NRTF1b, NRTF1c and NRTF1d), inositol 5-phosphatase (IPP), caffeic acid 7-O-methyltransferase (7OM), adenosine-5′-phosphate deaminase (AMPD), and two nematode responsive proteins (NRP) are provided. A CDPKa nucleotide encoding sequence is provided at SEQ ID: NO:1 with the corresponding protein at SEQ ID NO:2, a CDPKb nucleotide encoding sequence is provided at SEQ ID: NO:3 with the corresponding protein at SEQ ID NO:4, an NRTF1a nucleotide encoding sequence is provided at SEQ ID: NO:6 with the corresponding protein at SEQ ID NO:7 an NRTF1b nucleotide encoding sequence is provided at SEQ ID: NO:8 with the corresponding protein at SEQ ID NO:9 an NRTF1c nucleotide encoding sequence is provided at SEQ ID: NO:10 with the corresponding protein at SEQ ID NO:11, an NRTF1d nucleotide encoding sequence is provided at SEQ ID: NO:12 with the corresponding protein at SEQ ID NO:13, an NRP-1 nucleotide encoding sequence is provided at SEQ ID: NO:14 with the corresponding protein at SEQ ID NO:15, an NRP-2 nucleotide encoding sequence is provided at SEQ ID: NO:16 with the corresponding protein at SEQ ID NO:17, a 7OM nucleotide encoding sequence is provided at SEQ ID: NO:18 with the corresponding protein at SEQ ID NO:19, and an IPP nucleotide encoding sequence is provided at SEQ ID: NO:20 with the corresponding protein at SEQ ID NO:21. An adenosine-5′-phosphate deaminase (AMPD) gene is provided at SEQ ID NO: 33 with the corresponding protein at SEQ ID NO: 34. As discussed in more detail below, the sequences of the invention are involved in many basic biochemical pathways that are relevant to plant pathogen resistance. Thus, methods are provided for the expression, over-expression or co-suppression of these sequences in a host plant to modulate plant defense responses. Some of the methods involve stably transforming a plant with a nucleotide sequence capable of modulating the plant metabolism operably linked with a promoter capable of driving expression of a gene in a plant cell.

The compositions also comprise nucleic acid molecules comprising sequences useful in the control of gene expression in improving nematode resistance. Promoter and other regulatory elements which are natively associated with these genes are also provided or can be easily isolated using the sequences and methods described herein with no more than routine experimentation. These sequences can also be used to identify other promoters, or enhancer or other cis-acting elements in the regulatory regions of these promoter sequences. These regulatory elements provide for temporal and spatial expression of operably linked sequences with pathogen infection in a plant. Particularly, provided are soybean adenosine-5′-phosphate deaminase (hereinafter AMPD) and IPP regulatory regions, which are set forth in SEQ ID NOs:5 and 22, respectively. Methods are provided for the regulated expression of a nucleotide sequence of interest that is operably linked to the promoter regulatory sequences disclosed herein. Nucleotide sequences operably linked to the promoter sequences are transformed into a plant cell. Exposure of the transformed plant to a stimulus such as pathogen infection induces transcriptional activation of the nucleotide sequences operably linked to these promoter regulatory sequences.

The promoter sequences of the invention may find use in the regulated expression of an operably linked heterologous gene of interest. For example, the provided sequences may find use as a nematode-regulated promoter, such as a nematode-inducible promoter. In addition to these promoter sequences, the nematode-regulated promoters of the invention encompass fragments and variants of these particular promoters as defined herein. Thus, a fragment of the promoter sequences provided in SEQ ID NO:5 or SEQ ID NO:22 may be used either alone or in combination with other sequences to create synthetic promoters. In such embodiments, the fragments (also called “cis-acting elements” or “subsequences”) confer desired properties on the synthetic promoter, such as conferring increased transcription of operably linked sequences in response to stress caused by pathogen attack.

By “nematode-regulated” promoter is intended a promoter whose transcription initiation activity is either induced or repressed in response to a nematode or other pathogen stimulus. Thus, a nematode-inducible promoter increases expression of an operably linked nucleotide sequence in the presence of a nematode stimulus. In contrast, a nematode repressible promoter decreases the transcription of an operably linked nucleotide sequence in the presence of a nematode stimulus. Nematode-regulated promoters provide a means for improved regulation of genetically engineered nematode resistance in plants. In addition to these promoter sequences, the nematode-regulated promoters of the invention encompass fragments and variants of these particular promoters as defined herein. It is known that expression of a toxin gene product in nematode feeding sites can potentially harm uninfected plant cells in tissues adjacent to those sites. Thus, it can be beneficial to additionally alter the transgenic plant to express a product that counteracts excessive production of the toxin. See, for example, the methods disclosed in WO 92/21757.

Thus, in another embodiment of the invention, a nematode-repressible promoter is used in combination with a nematode-inducible promoter to effect improved regulation of nematode resistance in a plant. In this manner, two transgene units in one or two nucleic acid molecules are used in concert to transform plant cells and regenerate transgenic plants having improved nematode resistance with respect to nontransgenic plants of the same species. The first transgene unit comprises a nematode-inducible promoter operably linked to a nematode-resistance sequence. The second nucleic acid molecule comprises a nematode-repressible promoter operably linked to a heterologous nucleotide sequence that encodes a gene product that, when expressed in a plant cell, inhibits or inactivates a toxic product of a nematode-resistance gene (i.e., that encoded by the first nucleic acid molecule) that has been engineered within the plant cell.

“Nematodes,” as defined herein, refers to parasitic nematodes such as cyst, root knot, and lesion nematodes, including Heterodera spp, Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); Globodera rostochiensis and Globodera pallida (potato cyst nematodes). Other examples of nematodes and similar pathogens contemplated in the present invention are given elsewhere herein.

Thus, the nematode-inducible and nematode-repressible synthetic promoter sequences disclosed herein, when assembled within a nucleic acid molecule such that the promoter is operably linked to a heterologous nucleotide sequence of interest, enable expression or repression (inhibition) of expression of the heterologous nucleotide sequence in the cells of a plant stably transformed with this nucleic acid molecule. By “heterologous nucleotide sequence” is intended a sequence that is not naturally occurring with the promoter sequence. While this nucleotide sequence is heterologous to the promoter sequence, it may be homologous, native, heterologous, or foreign, to the plant host. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The type of nucleotide sequence within a nucleic acid molecule of the invention depends upon its intended use. Thus, when the nucleic acid molecule comprises a nematode-inducible promoter, it is of interest to operably link that promoter to a nucleotide sequence useful in improving pathogen resistance, more particularly in improving nematode resistance. Such sequences are referred to herein as “nematode-resistance sequence.” By “nematode-resistance sequence” is intended a sequence coding for an RNA and/or a protein or polypeptide that, when expressed, either inhibits, prevents, or repels nematode infection or invasion of a plant cell or the nematode growth and development within plant tissues, thereby limiting the spread and reproduction of the nematode. Such sequences include sequences encoding nematode-resistance proteins and cytotoxic proteins or polypeptides that disrupt cell metabolism, the byproducts of which are essential for nematode survival and/or reproduction. Expression of such sequences allows a plant to avoid the disease symptoms associated with nematode infections, or prevent or minimize nematodes from causing disease and associated disease symptoms. These sequences may function as nematicides, that is as nematode-killing sequences. Such killing may occur by direct action on nematodes, or by action on the cells of the plant on which the nematodes feed to kill those cells, thereby depriving the infecting nematodes of a site of entry or of feeding. Alternatively, such nematicides may act on other surrounding tissue to cause the release of nematode toxins from that tissue. Such nematode-resistance sequences are provided in, for example, U.S. Pat. Nos. 5,750,386; 5,994,627; 6,006,470; and 6,228,992, incorporated herein by reference. Other examples of nematode resistance genes include Oryzacystatin-1 and cowpea trypsin inhibitor (Urwin et al. (1998) Planta 204: 472-479); Rhg (Webb et al. (1995) Theor. Appl. Genet. 91: 574-581); Hsl (Cai et al. (1997) Science 275: 832-834); CRE3 (Lagudah et al. (1997) Genome 40: 650-665); all of which are herein incorporated by reference.

Structural genes employed in carrying out the present invention encode a product which is toxic to plant cells. A wide variety of protein or peptide products which are toxic to plant cells, and examples of nematode-resistance sequences that code for cytotoxic substances include, but are not limited to, enzymes capable of degrading nucleic acids (DNA, RNA) such as nucleases, restriction endonucleases (such as EcoRI), micrococcal nucleases, RNase A, and barnase (i.e., mature Bacillus amyloliquefaciens RNase; Mariani et al. (1990) Nature 347: 737-741 and Paddon and Hartley (1985) Gene 40: 231-39); enzymes which attack proteins such as proteases, trypsin, pronase A, carboxypeptidase, endoproteinase Asp-N, endoproteinase Glu-C, and endoproteinase Lys-C; ribonucleases such as RNase CL-3 and RNase T₁; toxins from plant pathogenic bacteria such as phaseolotoxin, tabtoxin, and syringotoxin; lipases such as produced from porcine pancreas and Candida cyclindracea, membrane channel proteins such as glp F and connexins, (gap junction proteins), and antibodies which bind proteins in the cell so that the cell is thereby killed or debilitated. Genes that produce antibodies to plant cell proteins can be produced as described in Huse et al. ((1989) Science 246: 1275-1281). Proteins to which such antibodies can be directed include, but are not limited to, RNA polymerase, respiratory enzymes, cytochrome oxidase, Krebs cycle enzymes, protein kinases, aminocyclopropane-l-carboxylic acid synthase, and enzymes involved in the shikimic acid pathway such as enolpyruvyl shikimic acid-5-phosphate synthase. The toxic product may either kill the plant cell in which it is expressed or simply disable the cell so that it is less capable of supporting the pathogen. Where the plant is a food plant, the plant-toxic product may be non-toxic to animals and/or humans. Of course the genes of the invention may function as structural genes, namely nucleic acid sequences encoding CDPK, NRTF1, IPP, 7OM, AMPD and NRP proteins.

In one embodiment, the toxic product is a structural gene encoding mature Bacillus amyloliquefaciens RNase (or Bamase). See, e.g., Mariani et al. (1990, supra); Paddon and Hartley (1985, supra). The toxic product may either kill the plant cell in which it is expressed or simply disable the cell so that it is less capable of supporting the pathogen. Where the plant is a food plant, the plant-toxic product may be non-toxic to animals and/or humans. Where the expression product of the structural gene is to be located in a cellular compartment other than the cytoplasm, the structural gene may be constructed to include regions which code for particular amino acid sequences which result in translocation of the product to a particular site, such as the cell plasma membrane, or may be secreted into the periplasmic space or into the external environment of the cell. Various secretory leaders, membrane integration sequences, and translocation sequences for directing the peptide expression product to a particular site are described in the literature. See, for example, Cashmore et al. (1985) Bio/Technology 3: 803-808; Wickner and Lodish (1985) Science 230: 400-407.

Nucleic acid sequences encoding gene products useful in improving resistance to nematodes and other pathogens are provided. Particularly, nucleic acid sequences encoding soybean CDPKs, NRTF1s, IPP, 7OM, AMPD and NRPs are provided. The CDPK, NRTF1, IPP, 7OM, AMPD and NRP genes and their promoter/regulatory regions are part of the plant's response to attack by nematodes and other pathogens. Thus the sequences of the invention find use in controlling or modulating gene expression as well as the response to nematode and other pathogen attack.

CDPKs are an important class of signaling proteins that are involved in many different signal transduction pathways. For example, CDPK may play an essential role in apoplastic oxidative burst in plant cells during plant-pathogen interactions. Calcium fluxes are essential for oxidative burst. Although the oxidative activity of peroxidase requires calcium, the fluxes have other functions. These may include activation of release of substrate, and, through the activation of a CDPK, regulation of enzymes involved in phytoalexin and cell wall phenolic production such as PAL (Bolwell G P, et al. 1999. Free Radic Res 1999. Suppl:S137-45). A maize CDPK may be involved in germination and pollen tube growth (Estruch J J, et al. Proc Natl Acad Sci 1994: 91(19):8837-41).

The isolation of carbocyclic coformycin as the herbicidally active component from a fermentation of Saccharothrix species was described previously (Bush et al. (1993) Phytochemistry 32: 737-739). The primary mode of action of carbocyclic coformycin has been identified as inhibition of the enzyme AMPD (EC 3.5.4.6) following phosphorylation at the 5′ hydroxyl on the carbocyclic ring in vivo. Studies of pea (Pisum sativum L. var Onward) seedlings showed that the 5′-phosphate analog of carbocyclic coformycin is a potent, tight binding inhibitor of AMP deaminase isolated from the seedlings. It has been proposed that inhibition of AMP deaminase leads to the death of the plant through perturbation of the intracellular ATP pool (Dancer J E, et al. (1997) Plant Physiol 114(1):119-29).

AP2 is a class of plant transcriptional factors that can regulate gene expression. The genes regulated by AP2 may be involved in disease resistance and stress tolerance. Using mRNA differential display analysis, Park et al.((2001) Plant Cell 13(5):1035-1046) isolated a salt-induced transcript that showed significant sequence homology with an EREBP/AP2 DNA binding motif from oilseed rape plants. With this cDNA fragment as a probe, Park et aL. (2001, supra) isolated a cDNA clone, Tsi1 (for Tobacco stress-induced gene1), from a tobacco cDNA library, which was found to be induced not only in NaCl-treated leaves but also in leaves treated with ethephon or salicylic acid. These results and others from Park et al. (2001, supra) suggest that Tsi1 might be involved as a positive trans-acting factor in two separate signal transduction pathways under abiotic and biotic stress.

The AP2/ERF-domain transcription factor ORCA3 is a master regulator of primary and secondary metabolism in Catharanthus roseus (periwinkle). Van der Fits and Memelink ((2001) Plant J (1):43-53) demonstrate that ORCA3 specifically binds to and activates gene expression via a previously characterized jasmonate- and elicitor-responsive element (JERE) in the promoter of the terpenoid indole alkaloid biosynthetic gene Strictosidine synthase (Str). ORCA3 mRNA accumulation was rapidly induced by the plant stress hormone methyl jasmonate and by a precursor and an intermediate of the jasmonate biosynthetic pathway, further substantiating the role for ORCA3 in jasmonate signaling. Van der Fits and Memelink (2001) (supra) conclude that ORCA3 regulates jasmonate-responsive expression of the Str gene via direct interaction with the JERE.

A tomato gene that is induced early after infection of tomato (Lycopersicon esculentum Mill.) with root-knot nematodes (Meloidogyne javanica) encodes a protein with 54% amino acid identity to miraculin, a flavorless protein that causes sour substances to be perceived as sweet (Brenner E D, (1998) et al. Plant Physiol; 118(1):237-47). This gene was therefore named LeMir (L. esculentum miraculin). Sequence similarity places the encoded protein in the soybean trypsin-inhibitor family. LeMir mRNA is found in root, hypocotyl, and flower tissues, with the highest expression in the root. Rapid induction of expression upon nematode infection is localized to root tips. In situ hybridization showed that LeMir is expressed constitutively in the root-cap and root-tip epidermis. Western-blot analysis showed that LeMir expression is up-regulated by nematode infection and by wounding. Immunoprint analysis revealed that LeMir is expressed throughout the seedling root, but that levels are highest at the root/shoot junction. Analysis of seedling root exudates revealed that LeMir is secreted from the root into the surrounding environment, suggesting that it may interact with soil-borne microorganisms.

Karrer et al. ((1998), Plant Mol Biol 36(5):681-90) used a functional screening method to isolate genes whose products elicit the hypersensitive response (HR) in response to plant pathogens. A cDNA library derived from tobacco leaves undergoing the HR was cloned into a tobacco mosaic virus (TMV)-based expression vector. Infectious transcripts were generated and used to inoculate tobacco plants lacking the N resistance gene (genotype Xanthi nn). Approximately 1/1000 of the infectious transcripts produced local lesions, and may thus elicit the HR. The cDNA inserts from 50 lesion-forming clones were recovered and sequenced. Comparisons with protein databases revealed homologies to (a) ubiquitin, (b) tobacco tumor-related protein, similar to Kunitz-type trypsin inhibitors and (c) ribosomal protein S14. Five clones were able to induce the expression of PR2, a gene which is specifically activated in the tobacco HR. Northern and western blot analyses of leaves infected by the clone encoding ubiquitin strongly suggest that the infection produced a co-suppression response. This observation supports the involvement of the ubiquitin system in the tobacco HR.

Caffeoyl-coenzyme A O-methyltransferase (CCoAOMT) methylates, in vitro, caffeoyl-CoA and 5-hydroxyferuloyl-CoA, two possible precursors in monolignol biosynthesis in vivo. (Meyermans H., et al. (2000) J Biol Chem 275(47):36899-909). Meyermans et al. clarified the in vivo role of CCoAOMT in lignin biosynthesis by generating transgenic poplars with 10% residual CCoAOMT protein levels in the stem xylem. Xylem analysis revealed that the affected transgenic lines had a 12% reduced Klason lignin content, an 11% increased syringyl/guaiacyl ratio in the noncondensed lignin fraction, and an increase in lignin-attached p-hydroxybenzoate but otherwise a lignin composition similar to that of wild type. Stem xylem of the CCoAOMT-down-regulated lines had a pink-red coloration, which coincided with an enhanced fluorescence of mature vessel cell walls. Feeding experiments showed that O(3)-beta-d-glucopyranosyl-caffeic acid and GSA are storage or detoxification products of caffeic and sinapic acid, respectively. The observation that down-regulation of CCoAOMT decreases lignin amount whereas GSA accumulates to 10% of soluble phenolics indicates that endogenously produced sinapic acid is not a major precursor in syringyl lignin biosynthesis.

The inositol polyphosphate 5-phosphatases are a family of enzymes that terminate signals generated by the phosphoinositide kinases and phospholipase C. Given the diverse signaling functions of both the polyphosphoinositides and Ins (1,4,5) P3, it is predicted that the 5-phosphatases play a critical role in regulating many cellular events, in particular membrane trafficking and cell growth (Mitchell C A, et al. (1996) Biochem Soc Trans 24(4):994-1000).

The internodal maize pulvinus responds to gravistimulation with differential cell elongation on the lower side. As the site of both graviperception and response, the pulvinus is an ideal system to study how organisms sense changes in orientation. Perera et al. (1999) Proc Natl Acad Sci 96(10): 5838-43 observed a transient 5-fold increase in inositol 1,4,5-trisphosphate (IP3) within 10 s of gravistimulation in the lower half of the pulvinus, indicating that the positional change was sensed immediately. Additionally, phosphatidylinositol 4-phosphate 5-kinase activity in the lower pulvinus half increased transiently within 10 min of gravistimulation, suggesting that the increased IP3 production was accompanied by an up-regulation of phosphatidylinositol 4,5-bisphosphate biosynthesis. Neither IP3 levels nor phosphatidylinositol 4-phosphate 5-kinase activity changed in pulvini halves from vertical control plants. The data presented by Perera et al. (1999, supra) indicate the involvement of IP3 and inositol phospholipids in both short- and long-term responses to gravistimulation.

Transformed plants can be obtained having altered or enhanced responses to nematode attack; hence, the methods and compositions may find uses in altering the response of plants to similar stresses as well. Thus, the sequences of the invention find use in engineering broad-spectrum disease and pest resistance in a variety of plants. A polypeptide is said to have CDPK, NRTF1, NRP, 7OM, AMPD or IPP activity when it has one or more of the properties of the native protein. It is within the skill in the art to assay protein activities obtained from various sources to determine whether the properties of the proteins are the same. In so doing, one of skill in the art may employ any of a wide array of known assays including, for example, biochemical and/or pathological assays. For example, one of skill in the art could readily produce a plant transformed with a CDPKa polypeptide variant and assay a property of native CDPKa protein in that plant material to determine whether a particular property of the native CDPK was retained by the variant.

The compositions and methods of the invention are involved in biochemical pathways and as such may also find use in the activation or modulation of expression of other genes, including those involved in other aspects of nematode or other pathogen response. For example, in one embodiment of the invention, the soybean AMPD promoter is used to drive expression of an insecticidal protein which is accordingly induced in response to wounding or damage to the root tissue.

Although there is some conservation among these genes, proteins encoded by members of these gene families may contain different elements or motifs or sequence patterns that modulate or affect the activity, subcellular localization, and/or target of the protein in which they are found. Such elements, motifs, or sequence patterns may be useful in engineering novel enzymes for modulating gene expression in particular tissues. By “modulating” or “modulation” is intended that the level of expression of a gene may be increased or decreased relative to genes driven by other promoters or relative to the normal or uninduced level of the gene in question.

These genes and promoter elements have all been shown to be regulated in response to nematode infection, CDPKa, NRTF1, and 7OM all were upregulated in response to nematode infection in a susceptible line but suppressed by SCN infection in an SCN resistant line. CDPKb, AMPD, and IPP were all up-regulated by SCN infection but the induced expression was much higher in the non resistant line compared to the expression in the resistant line. Thus these nematode-regulated genes and proteins can be used to modify a plant's response to nematode infection. According to the invention, transgenic plants expressing the proteins CDPKa and 7OM were found to have increased nematode infection rates. Thus inhibition of these genes and their products may be used to reduce nematode infection. Similarly, plants with the transgenes CDPKb, NRTF1a, and NRP were found to have reduced infection rates. Expression of the proteins encoded by the sequences of the invention can be used to modulate or regulate the expression of proteins in these pathogen-response pathways and other directly or indirectly affected pathways. Hence, the compositions and methods of the invention find use in altering plant response to the environment and environmental stimuli. In other embodiments, fragments of the genes are used to confer desired properties to synthetic protein constructs for use in regulating plant growth or cellular processes, such as root growth.

Co-suppression is the reduction in expression levels, usually at the level of RNA, of a particular endogenous gene or gene family by the expression of a homologous sense construct that is capable of transcribing mRNA of the same strandedness as the transcript of the endogenous gene (Napoli et al., Plant Cell 2:279-289 (1990); van der Krol et al., Plant Cell 2:291-299 (1990)). Co-suppression may result from stable transformation with a single copy nucleic acid molecule that is homologous to a nucleic acid sequence found within the cell (Prolls and Meyer, (1992) Plant J. 2:465-475) or with multiple copies of a nucleic acid molecule that is homologous to a nucleic acid sequence found within the cell (Mittlesten et al., (1994) Mol. Gen. Genet. 244:325-330). Genes, even though different, linked to homologous promoters may result in the co-suppression of the linked genes (Vaucheret, C. R. (1993) Acad. Sci. III 316:1471-1483; Flavell, (1994) Proc. Natl. Acad. Sci. (U.S.A.) 91:3490-3496; van Blokland et al. (1994) Plant J. 6:861-877; Jorgensen (1990) Trends Biotechnol. 8:340-344; Meins and Kunz, (1994) In: Gene Inactivation and Homologous Recombination in Plants, Paszkowski (ed.), pp. 335-348, Kluwer Academic, Netherlands).

The present invention provides for isolated nucleic acid molecules comprising nucleotide sequences encoding the amino acid sequence shown in SEQ ID NOs: 2, 4, 7, 9, 11, 13, 15, 17, 19, and 21 and their conservatively modified variants or the nucleotide sequences of the nucleic acid molecules deposited in a bacterial host as Patent Deposit No. PTA-4153. Further provided are polypeptides having an amino acid sequence encoded by a nucleic acid molecule described herein, for example those polypeptides comprising the sequences set forth in SEQ ID NOs: 2, 4, 7, 9, 11, 13, 15, 17, 19, and 21 or those deposited in a bacterial host as Patent Deposit No. PTA-4153, and fragments and variants thereof.

The present invention further provides for an isolated nucleic acid molecule comprising the sequences shown in SEQ ID NOs: 1, 3, 6, 8, 10, 12, 14, 16, 18, 20, or 22 or the nucleotide sequences deposited in a bacterial host as Patent Deposit No. PTA-4153.

Plasmids containing the nucleotide sequences of the invention were deposited with the Patent Depository of the American Type Culture Collection (ATCC), Manassas, Va., and assigned Patent Deposit No. PTA-4153. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. § 112.

The invention encompasses isolated or substantially purified nucleic acid or protein compositions. An “isolated” or “purified” nucleic acid molecule or protein, or biologically active portion thereof, is substantially free of other cellular material, or culture medium, when produced by recombinant techniques, or substantially free of chemical precursors or other chemicals when chemically synthesized. In some embodiments, an “isolated” nucleic acid is free of sequences (such as other protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, 0.4 kb, 0.3 kb, 0.2 kb, or 0.1 kb, or 50, 40, 30, 20, or 10 nucleotides that naturally flank the nucleic acid molecule in genomic DNA of the cell from which the nucleic acid is derived. A protein that is substantially free of cellular material includes preparations of protein having less than about 30%, 20%, 10%, or 5%, (by dry weight) of contaminating protein. When the protein of the invention or biologically active portion thereof is recombinantly produced, culture medium may represent less than about 30%, 20%, 10%, or 5% (by dry weight) of chemical precursors or non-protein-of-interest chemicals.

Fragments and variants of the disclosed nucleotide sequences are encompassed by the present invention. Fragments and variants of proteins encoded by the disclosed nucleotide sequences are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence affect development, developmental pathways, stress responses, and/or disease resistance by retaining CDPK, NRTF1, NRP, AMPD, 7OM, or IPP activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of a CDPK, NRTF1, NRP, 7OM, AMPD or IPP nucleotide sequence that encodes a biologically active portion of a CDPK, NRTF1, NRP, 7OM, AMPD or IPP protein of the invention will encode at least 12, 25, 30, 50, 75, 100, 125, 150, 175, 200, 225, 250, 275, 300, 325, 350, 375, 400, 425, 450, 475, 500, 525, 550, 575, 600, 625, 650, 675, or 680 contiguous amino acids, or up to the total number of amino acids present in a full-length CDPK, NRTF1, NRP, 7OM, AMPD or IPP protein of the invention (for example, 416 amino acids for SEQ ID NO:2).

Fragments of a CDPK, NRTF1, NRP, 7OM, AMPD or IPP nucleotide sequence that are useful as hybridization probes or PCR primers generally need not encode a biologically active portion of a protein. Thus, a fragment of a CDPK, NRTF1, NRP, 7OM, AMPD or IPP nucleotide sequence may encode a biologically active portion of a CDPK, NRTF1, NRP, 7OM, AMPD or IPP protein, or it may be a fragment that can be used as a hybridization probe or PCR primer using methods disclosed below. A biologically active portion of a CDPK, NRTF1, NRP, 7OM, AMPD or IPP protein can be prepared by isolating a portion of the CDPK, NRTF1, NRP, 7OM, AMPD or IPP nucleotide sequences of the invention, expressing the encoded portion (e.g., by recombinant expression in vitro), and assessing the activity of the resulting protein. Nucleic acid molecules that are fragments of a CDPK, NRTF1, NRP, 7OM, AMPD or IPP nucleotide sequence comprise at least 16, 20, 25, 30, 40, 50, 60, 70, 80, 90, 100, 125, 150, 175, 200, 225, 250, 300, 350, 400, 450, 500, 550, 600, 650, 700, 750, 800, 850, 900, 950, 1000, 1050, 1100, 1200, 1300, 1400, 1500, 1600, 1700, 1800, 1900, 2000, 2100, 2200, 2300, or 2400 nucleotides, or up to the number of nucleotides present in a full-length CDPK, NRTF1, NRP2, 7OM, AMPD or IPP nucleotide sequence disclosed herein (for example, 2322 nucleotides for SEQ ID NO: 1).

By “variants” is intended substantially similar sequences. For nucleotide sequences, conservative variants include those sequences that, because of the degeneracy of the genetic code, encode the amino acid sequence of one of the polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically-derived nucleotide sequences, such as those generated, for example, by using site-directed mutagenesis but which still encode a CDPK, NRTF1, NRP, 7OM, AMPD or IPP protein of the invention. Generally, variants of a particular nucleotide sequence of the invention will have at least 40%, 50%, 60%, 70%, generally at least 75%, 80%, 85%, or about 90%, 91%, 92%, 93%, 94%, 95%, 96%, 97%, 98%, 99%, or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, hence they will continue to possess at least one activity possessed by the native CDPK, NRTF1, NRP, 7OM, AMPD or IPP protein. Such variants may result from, for example, genetic polymorphism or from human manipulation. Biologically active variants of a CDPK, NRTF1, NRP, 7OM, AMPD or IPP native protein of the invention will have at least 40%, 50%, 60%, 70%, generally at least 75%, 80%, 85%, 86%, 87%, 88%, 89%, about 90%, 91%, 92%, 93%, 94%, 95%, 96%, 97%, 98%, 99%, or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6, as few as 5, as few as 4, 3, 2, or even 1 amino acid residue. As used herein, reference to a particular nucleotide or amino acid sequence (such as a CDPKa sequence) shall include all modified variants as described supra.

The proteins of the invention may be altered in various ways including amino acid substitutions, deletions, truncations, and insertions. Methods for such manipulations are generally known in the art. For example, amino acid sequence variants of the CDPK, NRTF1, NRP, 7OM, AMPD or IPP proteins of the instant invention can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be made.

Thus, the genes and nucleotide sequences of the invention include both naturally occurring sequences as well as mutant forms. Likewise, the proteins of the invention encompass both naturally-occurring proteins as well as variations and modified forms thereof. Such variants will continue to possess the desired CDPK, NRTF1, NRP, 7OM, AMPD or IPP activity. It is recognized that variants need not retain all of the activities and/or properties of the native CDPK, NRTF1, NRP, 7OM, AMPD or IPP protein. Obviously, the mutations that will be made in the DNA encoding the variant must not place the sequence out of reading frame and in some embodiments will not create complementary regions that could produce secondary mRNA structure. See, EP Patent Application Publication No. 0 075 444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity of CDPK, NRTF1, NRP, 7OM, AMPD or IPP polypeptides can be evaluated by either an enhanced response to nematode attack or a modulation in a plant developmental or metabolic process when expression of the protein or polypeptide sequence is altered. For example, CDPK, NRTF1, NRP, 7OM, AMPD or IPP activity may be evaluated as a change in gene transcription in genes downstream from CDPK, NRTF1, NRP, 7OM, AMPD or IPP in the nematode-response pathway in the plant.

Variant nucleotide sequences and proteins also encompass sequences and proteins derived from a mutagenic and recombinogenic procedure such as DNA shuffling. With such a procedure, one or more different CDPK, NRTF1, NRP, 7OM, AMPD or IPP coding sequences can be manipulated to create a new CDPK, NRTF1, NRP, 7OM, AMPD or IPP possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the CDPK, NRTF1, NRP, 7OM, AMPD or IPP genes of the invention and other known CDPK, NRTF1, NRP, 7OM, AMPD or IPP genes to obtain a new gene coding for a protein with an improved property of interest, such as an increased Km in the case of an enzyme. Strategies for such DNA shuffling are known in the art. See, for example, Stemmer (1994) Proc. Natl. Acad. Sci. USA 91:10747-10751; Stemmer (1994) Nature 370:389391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

These variant nucleotide sequences can also be evaluated by comparison of the percent sequence identity shared by the polypeptides they encode. For example, isolated nucleic acids which encode a polypeptide with a given percent sequence identity to the polypeptide of SEQ ID NO: 2, 4, 7, 9 and 11 are disclosed. Identity can be calculated using, for example, the BLAST, CLUSTALW, or GAP algorithms under default conditions. The percentage of identity to a reference sequence is at least 50% and, rounded upwards to the nearest integer, can be expressed as an integer selected from the group of integers consisting of from 50 to 99. Thus, for example, the percentage of identity to a reference sequence can be at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%.

The compositions of the invention also include isolated nucleic acid molecules comprising the promoter nucleotide sequences set forth in SEQ ID NOs:5 and 22. SEQ ID NO:5 sets forth the nucleotide sequence of the soybean AMPD promoter, while SEQ ID NO:22 sets forth the nucleotide sequence of the soybean IPP promoter. By “promoter” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate.

It is recognized that having identified the nucleotide sequences for the promoter regions disclosed herein, it is within the state of the art to isolate and identify additional regulatory elements in the 5′ untranslated region upstream from the particular promoter regions defined herein. Thus for example, the promoter regions disclosed herein may further comprise upstream regulatory elements that confer tissue-preferred expression of heterologous nucleotide sequences operably linked to the disclosed promoter sequence. See particularly, Australian Patent No. AU-A-77751/94 and U.S. Pat. Nos. 5,466,785 and 5,635,618. It is also recognized by those of skill in the art that regulatory elements may be found in transcribed regions of a gene, for example in the region between transcription start and translation start as well as 3′ to the end of translation; such elements may be found in the sequence set forth in SEQ ID NO:5 or 22. Regulatory elements, as used herein, may also be found within the coding region itself.

Fragments and variants of the disclosed AMPD or IPP promoter sequences are also encompassed by the present invention. By “fragment” is intended a portion of the nucleotide sequence. Fragments of a nucleotide sequence may retain biological activity and hence retain their transcriptional regulatory activity. Thus, for example, less than the entire promoter sequence disclosed herein may be utilized to drive expression of an operably linked nucleotide sequence of interest, such as a nucleotide sequence encoding a heterologous nematode-resistance polypeptide. Alternatively, fragments of a nucleotide sequence that are useful as hybridization probes generally do not retain biological activity. Thus, a fragment of AMPD or IPP promoter nucleotide sequence may encode a biologically active portion of the AMPD or IPP promoter, or it may be a fragment that can be used as a hybridization probe or PCR primer using methods disclosed below. A biologically active portion of an AMPD or IPP promoter can be prepared by isolating a portion of one of the AMPD or IPP promoter nucleotide sequences of the invention, and assessing the activity of the portion of the AMPD or IPP promoter. Nucleic acid molecules that are fragments of a AMPD or IPP promoter nucleotide sequence comprise at least about 16 to 20 nucleotides to about 30, 40, 50, 60, 70, 80, 90, 100, 150, 200, 250, 300, 350, 400, 450, 500, 550, 600, 650, 700, 750, 800, 850, 900, 950, 1000, 1050, 1100, 1200, 1300, 1400, 1500, 1600, 1700, 1800, 1900, or 2000 nucleotides, or up to the number of nucleotides present in a full-length AMPD or IPP nucleotide sequence disclosed herein (for example, 943 nucleotides for SEQ ID NO:5 and 1600 nucleotides for SEQ ID NO: 22).

Fragment lengths depend upon the objective and will also vary depending upon the particular promoter sequence. Thus, where the promoter fragment is to be used as a functional promoter, suitable promoter fragments or variants retain functional promoter activity, that is, the fragments or variants obtained are capable of directing RNA polymerase II to initiate RNA synthesis at the appropriate transcription initiation site for a particular coding sequence in response to a nematode stimulus where the promoter is nematode-inducible or direct transcription in the absence of the nematode stimulus in the case of a nematode-repressible promoter. It is within the skill in the art to determine whether such fragments decrease expression levels or alter the nature of expression, i.e., nematode-inducible or nematode-repressible expression, and assays to determine the activity of a promoter sequence are well known in the art. For example, the production of RNA transcripts may be assayed by northern blot hybridization. Alternatively, an AMPD or IPP promoter fragment or variant may be operably linked to the nucleotide sequence encoding any reporter protein, such as the (3-glucuronidase protein (GUS reporter) or the luciferase protein or the like. The DNA construct may inserted into the genome of a plant or plant cell and the mRNA or protein levels of the reporter sequence determined. See, for example, Eulgem et al. (1999) EMBO J 18: 4689-4699; U.S. Pat. No. 6,072,050, herein incorporated by reference.

By promoter “variants” is intended promoter sequences having substantial similarity with a synthetic promoter sequence disclosed herein. Generally, variants of a particular nucleotide sequence of the invention will have at least about 40%, 50%, 60%, 65%, 70%, generally at least about 75%, 80%, 85%, 86%, 87%, 88%, 89%, at least about 90%, 91%, 92%, 93%, 94%, 95%, 96%, 97%, or at least about 98%, 99% or more sequence identity to that particular nucleotide sequence as determined by sequence alignment programs described elsewhere herein using default parameters. Thus, variants may differ by only a few nucleotides, such as 50, 25, 20, 15, 10, 9, 8, 7, 6, 5, 4, 3, 2, or even 1 nucleotide. Such variants retain the nematode-regulated promoter activity of the disclosed promoter sequences. Thus variants of the AMPD or IPP sequence retain nematode-inducible promoter activity.

The variant promoter sequences will share substantial homology with their corresponding synthetic promoter sequence. By “substantial homology” is intended a sequence exhibiting substantial functional and structural equivalence with the disclosed sequence. Any functional or structural differences between substantially homologous sequences do not affect the ability of the sequence to function as a nematode-regulated promoter. Thus, for example, any sequence having substantial sequence homology with the sequence of a particular nematode-inducible promoter of the present invention will direct expression of an operably linked heterologous nucleotide sequence in response to a nematode stimulus. Two nucleotide sequences are considered substantially homologous when they have at least about 50%, 60%, 65%, 70%, 73%, 75%, 78%, 80%, 81%, 82%, 83%, 84%, 85%, 86%, 87%, 88%, 89%, 90%, 91%, 92%, 93%, 94%, 95%, 96%, 97%, to 98% or 99% or higher sequence homology. Substantially homologous sequences of the present invention include variants of the disclosed sequences such as those that result from site-directed mutagenesis, as well as synthetically derived sequences.

The nucleotide sequences of the invention can be used to isolate corresponding sequences from other organisms, particularly other plants, more particularly other crop plants. In this manner, methods such as PCR, hybridization, and the like can be used to identify such sequences based on their sequence homology to the sequences set forth herein. Sequences isolated based on their sequence identity to the nucleotide sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species. Thus, isolated sequences that have CDPK, NRTF1, NRP, 7OM, AMPD or IPP activity, sequences with AMPD or IPP promoter activity, or sequences which encode a CDPK, NRTF1, NRP, 7OM, AMPD or IPP protein and which hybridize under stringent conditions to the CDPK, NRTF1, NRP, 7OM, AMPD or IPP sequences disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present it a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the disease-resistant sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire sequence disclosed herein, or one or more portions thereof, may be used as a probe capable of specifically hybridizing to corresponding nematode-response sequences, including promoters and messenger RNAs. To achieve specific hybridization under a variety of conditions, such probes include sequences that are unique among nematode-response sequences and may be at least about 10 or 15 or 17 nucleotides in length or at least about 20 or 22 or 25 nucleotides in length. Such probes may be used to amplify corresponding sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different under different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length or less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3. Incubation should be at a temperature of least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl, 0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a final wash in 0.1×SSC at 60 to 65° C. for at least about 20 minutes. Optionally, wash buffers may comprise about 0.1% to about 1% SDS. Duration of hybridization is generally less than about 24 hours, usually about 4 to about 12 hours.

Specificity is typically a function of post-hybridization washes, the critical factors being the ionic strength and temperature of the final wash solution. For DNA-DNA hybrids, the Tₘ (thermal melting point) can be approximated from the equation of Meinkoth and Wahl ((1984) Anal. Biochem. 138:267-284): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), the SSC concentration may be increased so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

In general, sequences that have AMPD or IPP promoter activity or encode a CDPK, NRTF1, NRP, 7OM, AMPD or IPP protein and which hybridize to the CDPK, NRTF1, NRP, 7OM, AMPD, IPP, AMPD promoter or IPP promoter sequences disclosed herein will be at least about 40% homologous, about 50% or 60% homologous, about 70% homologous, and even about 80%, 85%, 90%, 91%, 92%, 93%, 94%, 95%, 98%, 99% or more homologous with the disclosed sequences. That is, the sequence identity of the sequences may be from about 40% to 50% identical, about 60% to 70% or 75%, and even about 80%, 85%, 87%, 90%, 91%, 92%, 93%, 94%, 95%, 96%, 97%, 98%, or 99% identical, or higher, so that the sequences may differ by only 10, 9, 8, 7, 6, 5, 4, 3, 2, or 1 amino acid residue or by 20, 19, 18, 17, 16, 15, 14, 13, 12, 11, 10, 9, 8, 7, 6, 5, 4, 3, 2, or 1 nucleic acid.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences.

Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 10 (available from Genetics Computer Group (GCG), Accelrys, Inc., San Diego, Calif.). Alignments using these programs can be performed using the default parameters. The CLUSTAL program is well described by Higgins et al. (1988) Gene 73:237-244 (1988); Higgins et al. (1989) CABIOS 5:151-153; Corpet et el. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences.

The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used (see information at www.ncbi.nlm.nih.gov). Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by GAP Version 10. GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989), Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 80%, 85%, 90%, 95%, or higher sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 50%, 55%, 60%, 65%, 70%, 75%, 80%, 85%, 90%, or at least 95% or higher sequence identity.

Another indication that nucleotide sequences are substantially identical is if two molecules hybridize to each other under stringent conditions. Generally, stringent conditions are selected to be about 5° C. lower than the Tₘ for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross-reactive with the polypeptide encoded by the second nucleic acid.

(e)(ii) The term “substantial identity” in the context of a peptide indicates that a peptide comprises a sequence with at least 70%, 75%, 80%, 83%, 85%, 88%, 90%, 93%, 95%, 96%, 97%, 98%, or 99% or higher sequence identity to the reference sequence over a specified comparison window. Preferably, optimal alignment is conducted using the homology alignment algorithm of Needleman and Wunsch (1970) J Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

Compositions and methods for improving resistance to nematodes are provided. The compositions comprise CDPK, NRTF1, NRP, 7OM, AMPD and IPP genes and proteins as well as AMPD and IPP promoters. Because resistance to nematodes involves the plant defense response, increased resistance to nematodes may well confer increased resistance to other plant pathogens and diseases. For example, it is recognized that expression driven by pathogen-responsive promoter regions can be influenced by more than one pathogen or pest (see, for example, Strittmatter et al. (1996) Mol. Plant Microb. Interact. 9: 68-73). Thus, in other embodiments of the invention, the nematode-regulated AMPD or IPP promoters can be used to create or enhance resistance of a plant to other pathogens or pests in accordance with the methods of the invention whenever infection by those pathogens or pests triggers enhanced or selective transcription from these promoters. Accordingly, the compositions and methods are useful in protecting plants against a broader spectrum of diseases and stress, including stress caused by the attack or infection of fungal pathogens, viruses, insects and the like. In some embodiments, the disease or stress or attack induces transcription from the AMPD or IPP promoters at the site of infection of the plant. In this manner, a nematode-regulated AMPD or IPP promoter, or variant or fragment thereof, can be operably linked to a nucleotide sequence that encodes pathogen-resistance sequences.

By “resistance” in the context of pathogen-resistance, disease-resistance, or nematode resistance is intended that the impact on the plant of the particular pathogen, disease, and/or nematode attack is diminished or entirely avoided. That is, in a plant showing resistance, pathogens are prevented from causing plant diseases and the associated disease symptoms, or alternatively, some or all of the disease symptoms caused by the pathogen are minimized or lessened. This includes but is not limited to the ability of a host ot prevent nematode reproduction. Genes encoding disease resistance traits include, generally, detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. In some embodiments, the expression of a CDPK, NRTF1, NRP, 7OM, AMPD or IPP gene product, either driven by an AMPD or IPP promoter or a heterologous promoter, may be induced in response to disease or stress or attack and confers disease resistance; i.e., production of the CDPK, NRTF1, NRP, 7OM, AMPD or IPP gene product lessens the symptoms that would ordinarily result in a plant.

The nucleic acid molecules of the present invention are useful in methods directed to creating or enhancing pathogen-resistance, more particularly nematode resistance in a plant. Improved pathogen-resistance may be accomplished by stably transforming a plant of interest with a nucleic acid molecule that comprises a nematode-regulated promoter identified herein operably linked to a pathogen-resistance sequence to produce antipathogenic activity in such plants, or by the use of such transformed plants or other products to produce antipathogenic compositions.

By “antipathogenic compositions” is intended that the compositions of the invention have antipathogenic activity and thus are capable of suppressing, controlling, and/or killing the invading pathogenic organism. An antipathogenic or nematicidal composition of the invention will reduce the disease symptoms resulting from pathogen or nematode challenge by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens and nematodes. In some embodiments, the pathogen-resistance sequence is a nematode-resistance sequence that, when expressed, produces a product that has antipathogenic properties for nematodes.

Assays that measure antipathogenic activity are commonly known in the art, as are methods to quantitate disease resistance in plants following pathogen infection. See, for example, U.S. Pat. No. 5,614,395, herein incorporated by reference. These assays may be used to measure the activity of the promoters of the invention as well as the activity of the polypeptides of the invention. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

Also contemplated are antipathogenic assays directed at nematode pathogens. Such assays are known to the skilled artisan, and may include assays directed at specific characteristics of nematode pathogen infections, such as assays directed at nematode feeding site formation. Such assays include those disclosed in U.S. Pat. Nos. 6,008,436; and 6,252,138, herein incorporated by reference.

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal and viral pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria altemata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines, Fusarnum solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarnum roseum, Alternaria altemata; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusanum, Xanthomonas campestris p.v. affalfae, Aphanomyces euteiches, Stemphylium herbarum, Stemphylium alfaffae; Wheat: Pseudomonas syringae p.v. atrofaciens, Urocystis agropyri, Xanthomonas campestris p.v. translucens, Pseudomonas syringae p.v. syringae, Alternaria altemata, Cladosporium herbarum, Fusarium graminearum, Fusarium avenaceum, Fusarium culmorum, Ustilago tritici, Ascochyta tritici, Cephalosponum gramineum, Collotetrichum graminicola, Erysiphe graminis f sp. tritici, Puccinia graminis f.sp. tritici, Puccinia recondita f.sp. tritici, Puccinia strifformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolans sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Tilletia indica, Rhizoctonia solani, Pythium graminicola, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium mondiforme, Alternaria altemata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium redianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola; Rice: rice brownspot fungus (Cochliobolus miyabeanus), rice blast fungus—Magnaporthe grisea (Pyricularia grisea), Magnaporthe salvinii (Sclerotium oryzae), Xanthomomas oryzae pv. oryzae, Xanthomomas oryzae pv. oryzicola, Rhizoctonia spp. (including but not limited to Rhizoctonia solani, Rhizoctonia oryzae and Rhizoctonia oryzae-sativae), Pseudomonas spp. (including but not limited to Pseudomonas plantarii, Pseudomonas avenae, Pseudomonas glumae, Pseudomonas fuscovaginae, Pseudomonas alboprecipitans, Pseudomonas syringae pv. panici, Pseudomonas syringae pv. syringae, Pseudomonas syringae pv. oryzae and Pseudomonas syringae pv. aptata), Erwinia spp. (including but not limited to Erwinia herbicola, Erwinia amylovaora, Erwinia chrysanthemi and Erwinia carotovora), Achyla spp. (including but not limited to Achyla conspicua and Achyla klebsiana), Pythium spp. (including but not limited to Pythium dissotocum, Pythium irregulare, Pythium arrhenomanes, Pythium myriotylum, Pythium catenulatum, Pythium graminicola and Pythium spinosum), Saprolegnia spp., Dictyuchus spp., Pythiogeton spp., Phytophthora spp., Alternaria padwickii, Cochliobolus miyabeanus, Curvularia spp. (including but not limited to Curvularia lunata, Curvularia affinis, Curvularia clavata, Curvularia eragrostidis, Curvularia fallax, Curvularia geniculata, Curvularia inaequalis, Curvularia intermedia, Curvularia oryzae, Curvularia oryzae-sativae, Curvularia pallescens, Curvularia senegalensis, Curvularia tuberculata, Curvularia uncinata and Curvularia verruculosa), Sarocladium oryzae, Gerlachia oryzae, Fusarium spp. (including but not limited Fusarium graminearum, Fusarium nivale and to different pathovars of Fusarium monoliforme, including pvs. fujikuroi and zeae), Sclerotium rolfsii, Phoma exigua, Mucor fragilis, Trichoderma viride, Rhizopus spp., Cercospora oryzae, Entyloma oryzae, Dreschlera gigantean, Sclerophthora macrospora, Mycovellosiella oryzae, Phomopsis oryzae-sativae, Puccinia graminis, Uromyces coronatus, Cylindrocladium scoparium, Sarocladium oryzae, Gaeumannomyces graminis pv. graminis, Myrothecium verrucaria, Pyrenochaeta oryzae, Ustilaginoidea virens, Neovossia spp. (including but not limited to Neovossia horrida), Tilletia spp., Balansia oryzae-sativae, Phoma spp. (including but not limited to Phoma sorghina, Phoma insidiosa, Phoma glumarum, Phoma glumicola and Phoma oryzina), Nigrospora spp. (including but not limited to Nigrospora oryzae, Nigrospora sphaerica, Nigrospora panici and Nigrospora padwickii), Epiococcum nigrum, Phyllostica spp., Wolkia decolorans, Monascus purpureus, Aspergillus spp., Penicillium spp., Absidia spp., Mucor spp., Chaetomium spp., Dematium spp., Monilia spp., Streptomyces spp., Syncephalastrum spp., Verticillium spp., Nematospora coryli, Nakataea sigmoidea, Cladosporium spp., Bipolaris spp., Coniothyrium spp., Diplodia oryzae, Exserophilum rostratum, Helococera oryzae, Melanomma glumarum, Metashaeria spp., Mycosphaerella spp., Oidium spp., Pestalotia spp., Phaeoseptoria spp., Sphaeropsis spp., Trematosphaerella spp., rice black-streaked dwarf virus, rice dwarf virus, rice gall dwarf virus, barley yellow dwarf virus, rice grassy stunt virus, rice hoja blanca virus, rice necrosis mosaic virus, rice ragged stunt virus, rice stripe virus, rice stripe necrosis virus, rice transitory yellowing virus, rice tungro bacilliform virus, rice tungro spherical virus, rice yellow mottle virus, rice tarsonemid mite virus, Echinochloa hoja blanca virus, Echinochloa ragged stunt virus, orange leaf mycoplasma-like organism, yellow dwarf mycoplasma-like organism, Aphelenchoides besseyi, Ditylenchus angustus, Hirschmanniella spp., Criconemella spp., Pratylenchus spp., Hoplolaimus indicus

Nematodes include parasitic nematodes such as root-knot, cyst, and lesion nematodes, including Heterodera and Globodera spp; particularly Globodera rostochiensis and globodera pallida (potato cyst nematodes); Heterodera glycines (soybean cyst nematode); Heterodera schachtii (beet cyst nematode); and Heterodera avenae (cereal cyst nematode).

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicomis barberi, northern corn rootworm; Diabrotica undecimpunctata howardi, southern corn rootworm; Melanotus spp., wireworms; Cyclocephala borealis, northern masked chafer (white grub); Cyclocephala immaculate, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicomis, corn blotch leafminer; Anaphothrips obscrurus, grass thrips; Solenopsis milesta, thief ant; Tetranychus urticae, two-spotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha (lava, yellow sugarcane aphid; Blissus leucopterus leucoptenus, chinch bug; Contarinia sorghicola, sorghum midge; Tetranychus cinnabarinus, carmine spider mite; Tetranychus urticae, two-spotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis grandis, boll weevil; Aphis gossypii, cotton aphid; Pseudatomoscelis seriatus, cotton fleahopper; Trialeurodes abutilonea, banded-winged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, two-spotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrostemum hilare, green stink bug; Sow: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrostemum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, two-spotted spider mite; Barley: Ostrinia nubilalis, European corn borer; Agrotis ipsilon, black cutworm; Schizaphis graminum, greenbug; Blissus leucopterus leucopterus, chinch bug; Acrostemum hilare, green stink bug; Euschistus servos, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

By “anti-pathogenic compositions” is intended that the compositions of the invention are capable of suppressing, controlling, and/or killing the invading pathogenic organism or insect pest.

Methods for increasing pathogen resistance in a plant are provided. In some embodiments, the methods involve stably transforming a plant with a DNA construct comprising an anti-pathogenic nucleotide sequence of the invention operably linked to a promoter that drives expression in a plant. While the choice of promoter will depend on the desired timing and location of expression of the anti-pathogenic or other nucleotide sequences, desirable promoters include constitutive and pathogen-inducible promoters. In some embodiments, such a promoter will be an AMPD or IPP promoter of the invention, as further discussed below. These methods may find use in agriculture, particularly in limiting the impact of plant pathogens or insect pests on crop plants. Thus, transformed plants, plant cells, plant tissues and seeds thereof are provided by the present invention.

Additionally, the compositions of the invention can be used in formulations for their disease resistance activities. The proteins of the invention can be formulated with an acceptable carrier into a pesticidal or nematicidal composition(s) that is, for example: a suspension, a solution, an emulsion, a dusting powder, a dispersible granule, a wettable powder, an emulsifiable concentrate, an aerosol, an impregnated granule, an adjuvant, a coatable paste, or an encapsulation in, for example, polymer substances.

It is understood in the art that plant DNA viruses and fungal pathogens remodel the control of the host replication and gene expression machinery to accomplish their own replication and effective infection. The plant response to stress, such as stress caused by nematode attack, is known to involve many basic biochemical pathways and cellular functions. Hence, the sequences of the invention may find use in altering the defense mechanisms of a host plant to provide broad-based resistance to disease or insect pests. Additionally, the present invention may be useful in preventing corruption of the cell machinery by viruses and other plant pathogens.

The compositions and methods of the invention function to inhibit or prevent plant diseases. The gene products may accomplish their anti-pathogenic effects by suppressing, controlling, and/or killing the invading pathogenic organism. Further, the promoters of the invention may provide control of gene expression that may be helpful in avoiding or ameliorating disease symptoms. It is recognized that the present invention is not dependent upon a particular mechanism of defense. Rather, the compositions and methods of the invention work to increase resistance of the plant to pathogens independent of how that resistance is increased or achieved.

The methods of the invention can be used with other methods available in the art for enhancing disease resistance in plants. Similarly, in addition to being used singly, the pathogen-resistance sequences, more particularly the nematode-resistance sequences, described herein may be used in combination with sequences encoding other proteins or agents to protect against plant diseases and pathogens. Other plant defense proteins include, but are not limited to, those described in U.S. Pat. Nos. 6,586,657; 6,476,292; and U.S. Application Ser. No. 09/256,158, filed Feb. 24, 1999, now abandoned, all of which are herein incorporated by reference.

The present invention may be used in conjunction with one or more other methods to increase disease resistance. In some embodiments of the invention, a second nucleotide sequence is transformed into a plant to increase the plant's resistance to pathogens or pests. In these embodiments, any one of a variety of second nucleotide sequences may be utilized. It is recognized that such second nucleotide sequences may be used in either the sense or antisense orientation.

In other embodiments, the methods of the present invention involve stably transforming a plant with a DNA construct comprising a promoter of the invention linked to a nucleotide sequence which confers increased resistance to pathogens or pests. In this manner, the AMPD and IPP promoters disclosed herein may provide regulation of expression of operably linked coding regions to control pathogen and insect pests. Additionally, the AMPD and IPP promoters disclosed herein are useful for genetic engineering of plants to express a phenotype of interest. The promoter sequences may be used to drive expression of any heterologous nucleotide sequence. Alternatively, the AMPD or IPP promoter sequence may be used to drive expression of its native, i.e., naturally occurring gene sequence, such as the IPP gene sequence disclosed herein. In such an embodiment, the phenotype of the plant is altered. In some embodiments, the AMPD or IPP promoter sequences are operably linked to a nematicidal nucleotide sequence and drive expression of said sequence in a plant cell. The AMPD or IPP promoter sequences may therefore be used in creating or enhancing pathogen, disease, or pest resistance in a transformed plant.

In some embodiments, the nucleic acid molecules comprising CDPK, NRTF1, NRP, 7OM, AMPD or IPP sequences of the invention are provided in expression cassettes or DNA constructs for expression in the plant of interest. Such cassettes will include 5′ and 3′ regulatory sequences operably linked to a CDPK, NRTF1, NRP, 7OM, AMPD or IPP sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be co-transformed into the organism. Alternatively, the additional gene(s) can be provided on multiple expression cassettes.

Such an expression cassette is provided with a plurality of restriction sites for insertion of the CDPK, NRTF1, NRP, 7OM, AMPD or IPP sequence to be under the transcriptional regulation of the regulatory regions. The expression cassette may additionally contain selectable marker genes. The expression cassette will include in the 5′-3′ direction of transcription, a transcriptional and translational initiation region, a CDPK, NRTF1, NRP, 7OM, AMPD or IPP DNA sequence of the invention, and a transcriptional and translational termination region functional in plants. The transcriptional initiation region, or promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a “chimeric gene” comprises a coding sequence operably linked to a transcription initiation region that is heterologous to the coding sequence.

While it may be preferable to express the CDPK, NRTF1, NRP, 7OM, AMPD or IPP sequences using heterologous promoters, the native promoter sequences may be used. Such constructs would change expression levels of the CDPK, NRTF1, NRP, 7OM, AMPD or IPP protein in the plant or plant cell. Thus, the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639. Where appropriate, the gene(s) may be optimized for increased expression in the transformed plant. That is, the genes can be synthesized using plant-preferred codons for improved expression. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well characterized sequences that may be deleterious to gene expression. The G-C content of the sequence may be adjusted to enhance expression in a given host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, Omega prime (the 5′-leader sequence of tobacco mosaic virus RNA, Nucleic Acids Res 1987 Apr. 24; 15(8):3257-73), EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Allison et al. (1986) Virology 154:9-20); MDMV leader (Maize Dwarf Mosaic Virus); and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In those instances where it is desirable to have the expressed product of the heterologous nucleotide sequence of interest directed to a particular organelle, such as the chloroplast or mitochondrion, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase, and the like.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention. Alternatively, nematode-resistance may be directly selected by inoculating nematodes into the transformed protoplasts, cells, or tissues. Both methods of selection are generally known in the art. A number of promoters can be used in the practice of the invention. The promoters can be selected based on the desired outcome. That is, the nucleic acids can be combined with constitutive, tissue-preferred, or other promoters for expression in plants. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Generally, it will be beneficial to express the gene from an inducible promoter, particularly from a pathogen-inducible promoter, such as UCP3, (see co-pending U.S. application Ser. No. 10/266,416) or a nematode-repressible promoters, such as SCP1 (see co-pending U.S. application Ser. No. 10/266,416). Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J Plant Pathol. 89:245-254; Uknes et al. (1992) Plant Cell 4:645-656; and Van Loon (1985) Plant Mol. Virol. 4:111-116. See also the co-pending applications entitled “Inducible Maize Promoters,” U.S. Pat. No. 6,429,362, herein incorporated by reference.

Of interest are promoters that are expressed locally at or near the site of pathogen infection or pest or insect damage. See, for example, Marineau et al. (1987) Plant Mol. Biol. 9:335-342; Matton et al. (1989) Molecular Plant-Microbe Interactions 2:325-331; Somssich et al. (1986) Proc. Natl. Acad. Sci. USA 83:2427-2430; Somssich et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, the expression of which is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIPI (Rohmeier et al. (1993) Plant Mol. Biol. 22:783-792; Eckelkamp et al. (1993) FEBS Letters 323:73-76); MPI gene (Corderok et al. (1994) Plant J 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to: the maize ln2-2 promoter, which is activated by benzenesulfonamide herbicide safeners; the maize GST promoter, which is activated by hydrophobic electrophilic compounds that are used as pre-emergent herbicides; and the tobacco PR-1 a promoter, which is activated by salicylic acid. Other chemical-regulated promoters of interest include steroid-responsive promoters. See, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced gene expression within a particular plant tissue. Tissue-preferred promoters include those disclosed in Yamamoto et al. (1997) Plant J 12(2):255-265; Kawamata et al. (1997) Plant Cell Physiol. 38(7):792803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression. Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, those disclosed in U.S. application Ser. No. 10/104,706 (Isoflavone synthase promoter); Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):1122 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a B-glucuronidase reporter gene and introduced into both the nonlegume Nicotiana tabacum and the legume Lotus corniculatus, and in both instances root-specific promoter activity was preserved. Leach and Aoyagi ((1991) Plant Science 79(1):69-76)) describe their analysis of the promoters of the highly expressed roIC and roID root-inducing genes of Agrobacterium rhizogenes. They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TRI′ gene, fused to nptll (neomycin phosphotransferase II), showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

Where low level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts per cell. Alternatively, it is recognized that weak promoters also include promoters that are expressed in only a few cells and not in others to give a total low level of expression. Where a promoter is expressed at unacceptably high levels, portions of the promoter sequence can be deleted or modified to decrease expression levels. Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142, and 6,177,611, herein incorporated by reference.

As used herein, “vector” refers to a molecule such as a plasmid, cosmid or bacterial phage for introducing a nucleotide construct and/or expression cassette into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral nucleic acids. Generally, such methods involve incorporating a nucleotide construct of the invention within a viral DNA or RNA molecule. It is recognized that the CDPK, NRTF1, NRP, 7OM, AMPD or IPP proteins of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931, herein incorporated by reference.

A variety of other transformation protocols are contemplated in the present invention. Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050, 5,879,918, 5,886,244, 5,932,782; Tomes et al. (1995) Plant Cell, Tissue, and Organ Culture: Fundamental Methods, eds. Gamborg and Phillips (Springer-Verlag, Berlin); McCabe et al. (1988) Biotechnology 6:923-926); and Lec transformation (WO 00/28058, published May 18, 2000). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al (1998) Theor. Appl. Genet. 96:319-324 (soybean); Datta et al. (1990) Biotechnology 8:736-740 (rice); Klein et al. (1988) Proc. Natl. Acad. Sci. USA 85:43054309 (maize); Klein et al. (1988) Biotechnology 6:559-563 (maize); U.S. Pat. Nos. 5,240,855, 5,322,783 and 5,324,646; Tomes et al. (1995, supra) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

The present invention may be used for transformation of any plant species, including, but not limited to, monocots and dicots. Examples of plant species of interest include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis). Plants of the present invention may be crop plants (for example, alfalfa, sunflower, Brassica, cotton, safflower, peanut, sorghum, wheat, millet, tobacco, etc.), corn or soybean plants.

Plants of particular interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava bean, lentils, chickpea, etc.

Other plants of interest that are susceptible to diseases caused by nematodes, and the corresponding nematodes of interest include: alfalfa: Ditylenchus dipsaci, Meloidogyne hapla, Meloidogyne incognita, Meloidogynejavanica, Pratylenchus spp., Paratylenchus spp., Xiphinema spp.; banana: Radopholus similis, Helicotylenchus multicinctus, Meloidogyne incognita, M. arenaria, M. javanica, Pratylenchus coffeae, Rotylenchulus reniformis; beans & peas: Meloidogyne spp., Heterodera spp., Belonolaimus spp., Helicotylenchus spp., Rotylenchulus reniformis, Paratrichodorus anemones, Trichodorus spp.; cassava: Rotylenchulus reniformis, Meloidogyne spp.; cereals: Anguina tritici (Emmer, rye, spelt wheat), Bidera avenae (oat, wheat), Ditylenchus dipsaci (rye, oat), Subanguina radicicola (oat, barley, wheat, rye), Meloidogyne naasi (barley, wheat, rye), Pratylenchus spp. (oat, wheat, barley, rye), Paratylenchus spp. (wheat), Tylenchorhynchus spp. (wheat, oat); chickpea: Heterodera cajani, Rotylenchulus reniformis, Hoplolaimus seinhorsti, Meloidogyne spp., Pratylenchus spp.; citrus: Tylenchulus semipenetrans, Radopholus similis, Radopholus citrophilus (Florida only), Hemicycliophora arenaria, Pratylenchus spp., Meloidogyne spp., Bolonolaimus longicaudatus (Florida only), Trichodorus, Paratnchodorus, Xiphinema spp.; clover: Meloidogyne spp., Heterodera trifolii; coconut: Rhadinaphelenchus cocophilus; coffee: Meloidogyne incognita (most important in Brazil), M. exigua (widespread), Pratylenchus coffeae, Pratylenchus brachyurus, Radopholus similis, Rotylenchulus reniformis, Helicotylenchus spp.; com: Pratylenchus spp., Paratrichodorus minor, Longidorus spp., Hoplolaimus columbus; cotton: Meloidogyne incognita, Belonolaimus longicaudatus, Rotylenchulus reniformis, Hoplolaimus galeatus, Pratylenchus spp., Tylenchorhynchus spp., Paratrichodorus minor, grapes: Xiphinema spp., Pratylenchus vulnus, Meloidogyne spp., Tylenchulus semipenetrans, Rotylenchulus reniformis; grasses: Pratylenchus spp., Longidorus spp., Paratrichodorus christiei, Xiphinema spp., Ditylenchus spp.; peanut: Pratylenchus spp., Meloidogyne hapla., Meloidogyne arenaria, Criconemella spp., Belonolaimus longicaudatus (in Eastern United States); pigeonpea: Heterodera cajani, Rotylenchulus renifonnis, Hoplolaimus seinhorsti, Meloidogyne spp., Pratylenchus spp.; pineapple: Paratrichodorus christiei, Criconemella spp., Meloidogyne spp., Rotylenchulus reniformis, Helicotylenchus spp., Pratylenchus spp., Paratylenchus spp.; potato: Globodera rostochiensis, Globodera pallida, Meloidogyne spp., Pratylenchus spp., Trichodorus primitivus, Ditylenchus spp., Paratrichodorus spp., Nacoabbus aberrans; rice: Aphelenchiodes besseyi, Ditylenchus angustus, Hirchmanniella spp., Heterodera oryzae, Meloidogyne spp.; small fruits: Meloidogyne spp., Pratylenchus spp., Xiphinema spp., Longidorus spp., Paratrichodorus christiei, Aphelenchoides spp. (strawberry); soybean: Heterodera glycines, Meloidogyne incognita, Meloidogyne javanica, Belonolaimus spp., Hoplolaimus columbus; sugar beet: Heterodera schachtii, Ditylenchus dipsaci, Meloidogyne spp., Nacobbus aberrans, Trichodorus spp., Longidorus spp., Paratrichodorus spp.; sugar cane: Meloidogyne spp., Pratylenchus spp., Radopholus spp., Heterodera spp., Hoplolaimus spp., Helicotylenchus spp., Scutellonema spp., Belonolaimus spp., Tylenchorhynchus spp., Xiphinema spp., Longidorus spp., Paratrichodorus spp.; tea: Meloidogyne spp., Pratylenchus spp., Radopholus similis, Hemicriconemoides kanayaensis, Helicotylenchus spp., Paratylenchus curvitatus; tobacco: Meloidogyne spp., Pratylenchus spp., Tylenchorhynchus claytoni, Globodera tabacum, Trichodorus spp., Xiphinema americanum, Ditylenchus dipsaci (Europe only), Paratrichodorus spp.; tomato: Pratylenchus spp., Meloidogyne spp.; tree fruits: Pratylenchus spp. (apple, pear, stone fruits), Paratylenchus spp. (apple, pear), Xiphinema spp. (pear, cherry, peach), Cacopaurus pestis (walnut), Meloidogyne spp. (stone fruits, apple, etc.), Longidorus spp. (cherry), Criconemella spp. (peach), and Tylenchulus spp. (olive).

It is recognized that with these nucleotide sequences, antisense constructions complementary to at least a portion of the messenger RNA (mRNA) for the CDPK, NRTF1, NRP, 7OM, AMPD or IPP sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, 85%, 90%, 95% or more sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, such as greater than about 65%, 75%, 85%, 95%, or higher sequence identity. See, U.S. Pat. Nos. 5,283,184 and 5,034,323; herein incorporated by reference. Posttranscriptional gene silencing may also result from the presence of RNA or double-stranded RNA which is thought to trigger cell-mediated degradation of homologous RNAs. See, for example, Matzke et al. (2001) Curr. Op. Genet. Dev. 11:221-227.

The nucleotide sequences of the AMPD and IPP promoters disclosed in the present invention, as well as variants and fragments thereof, are useful in the genetic manipulation of any plant when assembled with a construct such that the promoter sequence is operably linked to a nucleotide sequence encoding a heterologous protein of interest. In this manner, the nucleotide sequences of the AMPD and IPP promoters of the invention can be provided in expression cassettes along with heterologous nucleotide sequences for expression in the plant of interest. Such an expression cassette is provided with a plurality of restriction sites for insertion of the nucleotide sequence to be under the transcriptional regulation of the nematode-regulated promoter region. The expression cassette may additionally contain selectable marker genes.

The expression cassette will include in the 5′-to-3′ direction of transcription a transcriptional and translational initiation region comprising the nematode-regulated AMPD or IPP promoter (or variant or fragment thereof), a nucleotide sequence of interest which may be a heterologous nucleotide sequence or a CDPK, NRTF1, NRP, 7OM, AMPD or IPP sequence, and a transcriptional and translational termination region functional in plants. The termination region may be native with the transcriptional initiation region comprising one of the promoter nucleotide sequences of the present invention, may be native with the DNA sequence of interest, or may be derived from another source. Convenient termination regions are available from the Ti-plasmid of A. tumefaciens, such as the octopine synthase and nopaline synthase termination regions (see references cited herein above). An expression cassette comprising an AMPD or IPP promoter sequence may also contain features or modifications as described herein above for expression cassettes comprising nucleotide sequences of a CDPK, NRTF1, NRP, 7OM, AMPD or IPP coding region of the invention.

The expression cassette comprising the AMPD or IPP promoter sequence (or variant or fragment thereof operably linked to a heterologous nucleotide sequence of interest may also contain at least one additional nucleotide sequence for a gene to be cotransformed into the organism. Alternatively, the additional sequence(s) can be provided on another expression cassette.

The promoter for the AMPD or IPP gene may regulate expression of operably linked nucleotide sequences in an inducible manner. That is, expression of the operably linked nucleotide sequences in a plant cell is induced in response to a stimulus. By “stimulus” is intended: a chemical, which may be applied externally or may accumulate in response to another external stimulus; other stresses such as environmental stresses, including but not limited to drought, temperature, and salinity; or other factor such as a pathogen, which may, for example, induce expression as a result of invading a plant cell. For example, a nematode invading a plant cell may produce a stimulus.

Synthetic promoters are known in the art. Such promoters comprise upstream promoter elements (also referred to as “fragments” or “subsequences”) of one nucleotide sequence operably linked to at least one promoter element of another nucleotide sequence. In an embodiment of the invention, heterologous gene expression is controlled by a synthetic hybrid promoter comprising the AMPD or IPP promoter sequences of the invention, or a variant or fragment thereof, operably linked to upstream promoter element(s) from a heterologous promoter. Upstream promoter elements that are involved in the plant defense system have been identified and may be used to generate a synthetic promoter. See, for example, Rushton and Somssich (1998) Curr. Opin. Plant Biol. 1:311-315. Also, for example, the UAR of maize ubiquitin-1 promoter has DNA elements that up-regulate promoter activity in response to nematode stimulus (see co-pending Application No. 60/329,667, filed Oct. 16, 2001). These elements can be as small as 4 or 6 base pairs, and can regulate nematode-responsive activity of other promoters by cloning one or more copies of the element into the promoters. Alternatively, a synthetic AMPD or IPP promoter sequence may comprise duplications of upstream elements found within the AMPD or IPP promoter sequence. In order to increase transcription levels, enhancers may be utilized in combination with the promoter regions of the invention. Enhancers are nucleotide sequences that act to increase the expression of a promoter region. Enhancers are known in the art and include the SV40 enhancer region, the 35S enhancer element, and the like.

It is recognized that the promoter sequence of the invention may be used with its native AMPD or IPP coding sequences. A DNA construct comprising the AMPD or IPP promoter operably linked with its native gene, such as the IPP gene of the invention, may be used to transform any plant of interest to bring about a desired phenotypic change, such as enhanced disease resistance. Where the promoter and its native gene are naturally occurring within the plant, i.e., in soybean, transformation of the plant with these operably linked sequences also results in either a change in phenotype such as enhanced stress response or the insertion of operably linked sequences within a different region of the chromosome, thereby altering the plant's genome.

In another embodiment of the invention, expression cassettes will comprise a transcriptional initiation region comprising the AMPD or IPP promoter nucleotide sequences disclosed herein, or variants or fragments thereof, operably linked to the heterologous nucleotide sequence whose expression is to be controlled by the inducible promoter of the invention. The promoter nucleotide sequences and methods disclosed herein are useful in regulating expression of any heterologous nucleotide sequence in a host plant in order to vary the phenotype of a plant. Various changes in phenotype are of interest including modifying the composition and content of root cells in response to nematode attack, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,885,801; 5,885,802; 5,990,389; and 5,703,049; herein incorporated by reference. Another example is lysine and/or sulfur rich seed protein encoded by the soybean 2S albumin described in U.S. Pat. No. 5,850,016 and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and PCT/US97/20441, filed Oct. 31, 1997, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European corn borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); lectins (Van Damme et al. (1994) Plant Mol. Biol. 24:825); and the like. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bargene encodes resistance to the herbicide basta, while the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049; 5,885,801; 5,885,802; 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as P-Ketothiolase, PHBase (polyhydroxybutyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content. In one embodiment, the nucleic acids of interest are targeted to the chloroplast for expression. In this manner, where the nucleic acid of interest is not directly inserted into the chloroplast, the expression cassette will additionally contain a nucleic acid encoding a transit peptide to direct the gene product of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1.990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Larrippa et al. (1988) J Biol. Chem. 263:14996-14999). See also Von Heijne et al. (1991) Plant Mol. Biol. Rep. 9:104-126; Clark et al. (1989) J. Biol. Chem. 264:1754417550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

In addition, it is recognized that methods of the present invention do not depend on the incorporation of the entire nucleotide construct into the genome. Rather, the methods of the present invention only require that the plant or cell thereof is altered as a result of the introduction of the nucleotide construct into a cell. In one embodiment of the invention, the genome may be altered following the introduction of the nucleotide construct into a cell. or example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference. The following examples are offered by way of illustration and not by way of limitation.

### EXAMPLES

### Example 1

### Composition of CDNA Libraries; Isolation and Sequencing of cDNA Clones

cDNA libraries representing mRNAs from various soybean tissues were prepared. The characteristics of the libraries are described below.

TABLE 1
cDNA Libraries from Soybean

| Library Name | Library description |
|---|---|
| Srm | Soybean (Glycine max L.) root meristem |
| Src3c | Soybean (Glycine max L., Bell) 8 days old inoculated |
| | with eggs of Cyst nematode (Race 14) for 4 days |
| sgs1c | Soybean (Glycine max L.) seeds 4 hrs after |
| | germination |
| sgs3c | Soybean (Glycine max L.) seeds 25 hrs after |
| | germination |
| sr1 | Soybean (Glycine max L.) root library |
| Srr1c | Soybean (Glycine max L.) root library |

cDNA libraries may be prepared by any one of many methods available. The cDNAs of the instant invention were introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries were converted into plasmid libraries according to the protocol provided by Stratagene. Upon conversion, cDNA inserts were inserted into the plasmid vector pBluescript. In addition, the cDNAs may also be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts were in plasmid vectors, plasmid DNA were prepared from randomly picked bacterial colonies containing recombinant pBluescript plasmids, or the insert cDNA sequences were amplified via polymerase chain reaction using primers specific for vector sequences flanking the inserted cDNA sequences. Amplified insert DNAs or plasmid DNAs were sequenced in dye-primer sequencing reactions to generate partial cDNA sequences (expressed sequence tags or “ESTs”; see Adams et al., (1991) Science 252:1651-1656). The resulting ESTs were analyzed using a Perkin Elmer Model 377 fluorescent sequencer.

Full-insert sequence (FIS) data was generated utilizing a modified transposition protocol. Clones identified for FIS were recovered from archived glycerol stocks as single colonies, and plasmid DNAs were isolated via alkaline lysis. Isolated DNA templates were reacted with vector primed M13 forward and reverse oligonucleotides in a PCR-based sequencing reaction and loaded onto automated sequencers. Confirmation of clone identification was performed by sequence alignment to the original EST sequence from which the FIS request is made.

Confirmed templates were transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system placed unique binding sites randomly throughout the population of large DNA molecules. The transposed DNA was then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones were randomly selected from each transposition reaction, plasmid DNAs were prepared via alkaline lysis, and templates were sequenced (ABI Prism dye-terminator ReadyReaction mix) outward from the transposition event site, utilizing unique primers specific to the binding sites within the transposon.

Sequence data was collected (ABI Prism Collection) and assembled using Phred/Phrap (P. Green, University of Washington, Seattle). Phred/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies were viewed by the Consed sequence editor (D. Gordon, University of Washington, Seattle).

### Example 2

### Identification of cDNA Clones

The genes of the invention were identified by searching a DuPont soybean EST database using the sequence information in the public domain. The main references used were Hermsmeier et al. (2000). Mol Plant Microb Interact 13:309-315; Hermsmeier et al. (1998). Mol Plant Microb Interact 11: 1258-1263; and Brenner et al., (1998). Plant Physiol. 118: 237-247.

cDNA clones encoding CDPK, AMPD, NRTF1, NRP, 7OM, and IPP were identified by conducting BLAST (Basic Local Alignment Search Tool; Altschul et al. (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis were compared to the GenBank database as described above. ESTs that contained sequences more 5- or 3-prime were found by using the BLASTn algorithm (Altschul et al (1997) Nucleic Acids Res. 25:3389-3402) against the DuPont proprietary database comparing nucleotide sequences that share common or overlapping regions of sequence homology. Where common or overlapping sequences existed between two or more nucleic acid fragments, the sequences were assembled into a single contiguous nucleotide sequence, thus extending the original fragment in either the 5 or 3 prime direction. Once the most 5-prime EST was identified, its complete sequence was determined by Full Insert Sequencing as described in Example 1. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

The cDNA clones were sequenced using an Applied Biosystems 373A (ABI) automated sequencer. Multiple-sequence alignment (Clustal W) and sequence similarity/identity (GCG and/or GAP) as well protein domain (BLOCKS) analyses were carried out using Curatool (CuraGen).

### Example 3

### Sequence and Gene Expression Analysis

To study the gene expression in SCN-soybean early interaction and to confirm the gene expression patterns, SCN infected and uninfected soybean tissues were prepared, RNA was isolated, and quantitative RT-PCR analysis was performed.

Soybean SCN resistant variety “Bell” was selected ((1990) Crop Science, Vol. 30: 1364-1365) for the experiment, along with susceptible varieties Pioneer Brand 9305 and 9281. Six seeds of 9281, 9305 and Bell were planted in each cup. A total of 48 cups were planted for the 48- and 96-hour samples and were inoculated using SCN race 3 eggs. For 24-hour samples, 1000 of J2 were used for inoculating each cup instead of SCN eggs (females).

The infected soybean roots were processed for the collection of SCN race 3 females. Females were treated in 0.01% HgCl₂ for 6 min. on a shaker, then rinsed with 0.1% Triton X-100. Surface sterilized females were rinsed in sterile, deionized water. Females were broken with a motorized pestle. Eggs were collected and counted. SCN J2 were hatched in 3.14 mM solution for 4 days and collected for inoculation.

42,000 eggs were pipetted into 3 holes for each infected sample (4-5 plants/sample)—4 cups per sampling time (48 hr and 96 hr) for each of the three varieties (24 sample/cups total). Noninfected samples/cups were not disturbed. For J2 inoculation of 24-hour samples, 1000 J2 were used for inoculating each cup instead of SCN eggs. The control plants were treated with water.

Total RNA was isolated from infected and uninfected soybean root samples after 24, 48 and 96 hours post-inoculation using Tripure according to manufacturer's protocol (Boehringer Mannheim, Indianapolis, Ind.). After removing genomic DNA with DNase I, 50 ng of the treated RNA were used for each RT-PCR reaction. The samples from the RT-PCR reaction were analyzed by gel electrophoresis to determine the expression levels of 7OM and CDPKa in the infected and uninfected samples of susceptible (S, 9281) and resistant (R, Bell) varieties at 24 hours after inoculation. To quantify the expression levels, the intensity of each PCR band was captured an analyzed by Alphalmager 2000 (Alpha Innotech Corporation). The expression ratio of each gene in susceptible (9281) and resistant (Bell) interactions was calculated by comparing data from uninfected with infected samples, giving the positive or negative values as shown in the fourth and fifth columns of Table 2.

TABLE 2
Effects of SCN race 3 infection on gene expression.

| Gene name | Symbol | SEQ ID NO: | 9281-24 H | BELL-24 H | Pattern |
|---|---|---|---|---|---|
| Calcium-dependent protein kina [symbol] | CDPKa | 1 | 0 | −3 | A |
| Calcium-dependent protein kina [symbol] | CDPKb | 3 | +1.42 | 0 | B |
| AP2-like protein (NRTF1) | NRTF1a | 6 | +1.07 | −1.71 | A |
| AMP deaminase | AMPD | 33 | −10.43 | +1.74 | B |
| Inositol 5-phosphatase | IPP | 20 | +7.56 | +2.11 | B |

As indicated in the Table 2, the expression patterns of the tested genes can be classified into groups A and B. In group A, gene expression was up-regulated by SCN infection in susceptible line 9281, whereas it was suppressed by SCN infection in resistant line Bell. In group B, gene expression was up-regulated by SCN infection, but the induced expression level is much higher in 9281 than in Bell.

Two soybean CDPK homologues were isolated and found to have different responsiveness to nematode infection in soybeans (Table 2). They have 51% similarity and 51% identity at the DNA sequence level, and 53% similarity and 41% identity at the amino acid sequence level. They also have high similarity with maize CDPK (L27484) and Arabidopsis thaliana CDPK (U20388) (Table 3).

TABLE 3
Percent similarity and identity (in parenthesis) between soybean and
other CDPKs at amino acid sequence level.

| | CDPKa | | |
|---|---|---|---|
| | (SEQ ID NO: 2) | U20388 | L27484 |
| CDPKa | | 53 (41) | 52 (40) |
| (SEQ ID NO: 2) | | | |
| CDPKb | 53 (41) | 81 (74) | 73 (65) |
| (SEQ ID NO: 4) | | | |

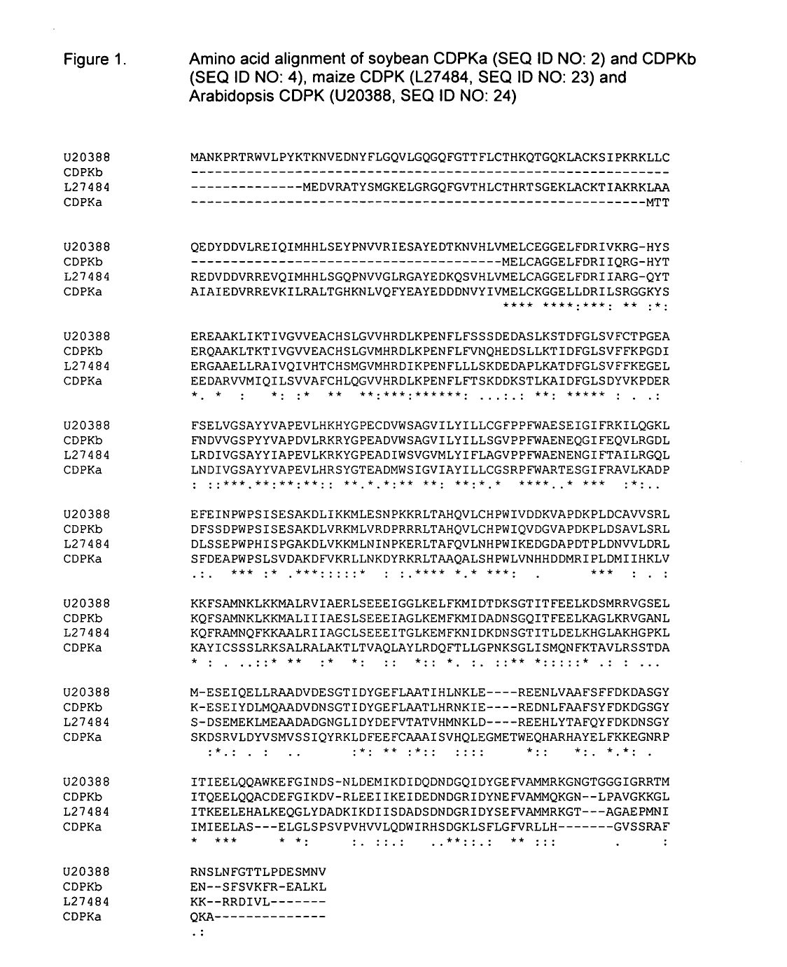

FIG. 1 shows the amino acid sequence alignment of soybean CDPKa (SEQ ID NO: 2), CDPKb (SEQ ID NO: 4), maize CDPK (L27484), and Arabidopsis CDPK (U20388).

Protein domain analysis indicates that both soybean CDPKa (amino acid residue 145 to 160) and CDPKb (amino acid residues 102 to 117) have a protein kinase ATP-binding domain (Ueda K. et al.; Gene 169:91-95(1996).

The information gathered from the gene expression study of soybean CDPKa and CDPKb indicates that both genes regulate the defense responses (both structural and metabolic reactions) to pathogen attack and/or formation of nematode feeding sites. Therefore, manipulation of their expression in transgenic plants can impact resistance to nematodes and other pathogens.

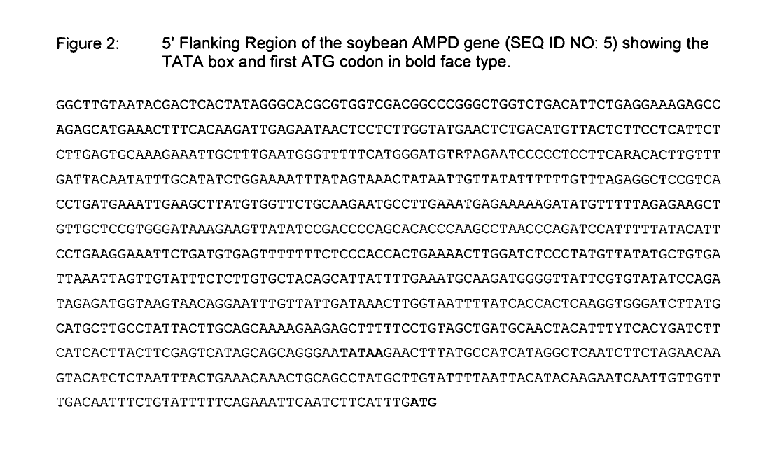

FIG. 2 is the 5′-flanking region of AMPD gene (SEQ ID NO: 5). The first MET codon and potential TATA box are shown in bold-face type.

The soybean AMPD gene sequence is disclosed in patent application WO 0109305-A. As shown in Table 1, AMPD gene expression was significantly up-regulated in 9281, indicating that the AMPD promoter is apparently a nematode-inducible promoter. The promoter can be used to express nematode resistance genes to engineer nematode resistance in plants. The promoter may have positive and negative cis-acting elements that mediate the various nematode-induced signals, and these elements may be used to synthesize or modify nematode-regulated promoters.

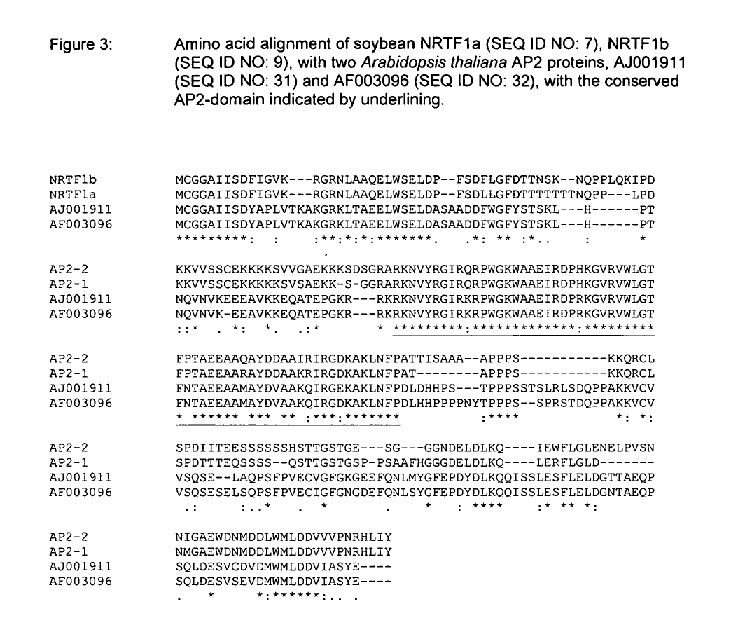

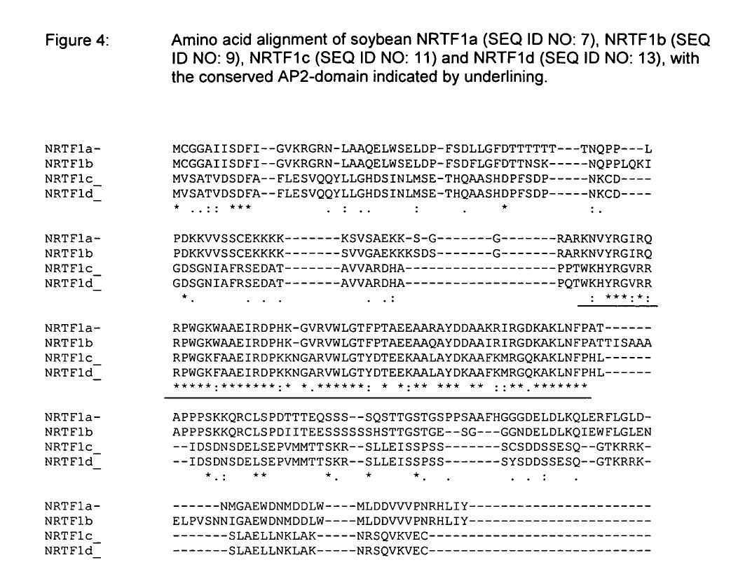

FIG. 3 shows the amino acid sequence alignment of soybean NRTF1a, NRTF1b, and two Arabidopsis AP2 proteins (SEQ ID NOs: 31 and 32, Accession Nos: AJ001911 and AF003096). FIG. 4 shows the amino acid sequence alignment of soybean NRTF1a, NRTF1b, NRTF1c, and NRTF1d proteins (SEQ ID NOs: 7, 9, 11, and 13). In both FIGS. 3 and 4, the conserved AP2-domain is indicated by underlining

The information disclosed herein obtained from various experiments indicates that soybean genes NRTF1a through d (SEQ ID NOs: 6, 8, 10, and 12) regulate the defense responses (both structural and metabolic reactions) to pathogen attack and/or the formation of nematode feeding sites. Therefore manipulation of their expression in transgenic plants can impact resistance to nematode and other pathogens.

TABLE 4
Percent similarity and identity (in parentheses) among
soybean NRTF1 genes at the amino acid level

| | NRTF1a | NRTF1b | NRTF1c | NRTF1d |
|---|---|---|---|---|
| NRTF1a | 100 (100) | 91 (89) | 70 (66) | 70 (66) |
| (SEQ ID NO: 7) | | | | |
| NRTF1b | | 100 (100) | 66 (61) | 53 (50) |
| (SEQ ID NO: 9) | | | | |
| NRTF1c | | | 100 (100) | 99 (99) |
| (SEQ ID NO: 11) | | | | |
| NRTF1d | | | | 100 (100) |
| (SEQ ID NO: 13) | | | | |

TABLE 5
Similarity and identity (in parenthesis) between soybean and other
tomato and tobacco NRP homologues at amino acid sequence level,
including tomato miraculin homologue (T07871), tobacco tumor-
related protein (T03803), rice alpha-amylase inhibitor (RASI) and a
potato trypsin inhibitor (U20592).

| | NRP-1 | T03803 | T07871 | RASI | U20592 |
|---|---|---|---|---|---|
| NRP-1 | | 58 (51) | 59 (52) | 44 (36) | 43 (33) |
| (SEQ ID NO: 15) | | | | | |
| NRP-2 | 63 (56) | 59 (54) | 61 (54) | 42 (37) | 42 (33) |
| (SEQ ID NO: 17) | | | | | |

As indicated in Table 5, the soybean NRP proteins of the invention have relatively high homology to a rice alpha-amylase inhibitor (RASI) (Ohtsubo, K.-l and Richardson, M. (1992) FEBS Lett 309: 68-72) and potato trypsin inhibitor (U20592) (Milligan, S. B. and Gasser, C. S. (1995) Plant Mol. Biol. 28(4), 691-711). Trypsin activity has been detected in nematode intestines (Lilley, C. J. et al. 1996. Parasitology. 113: 415-424) and overexpression of trypsin inhibitor can confer nematode resistance (Urwin, P. E. et al. (1998) Planta. 204: 472-479). Therefore overexpression of the NRP proteins of the invention can confer nematode resistance in planta.

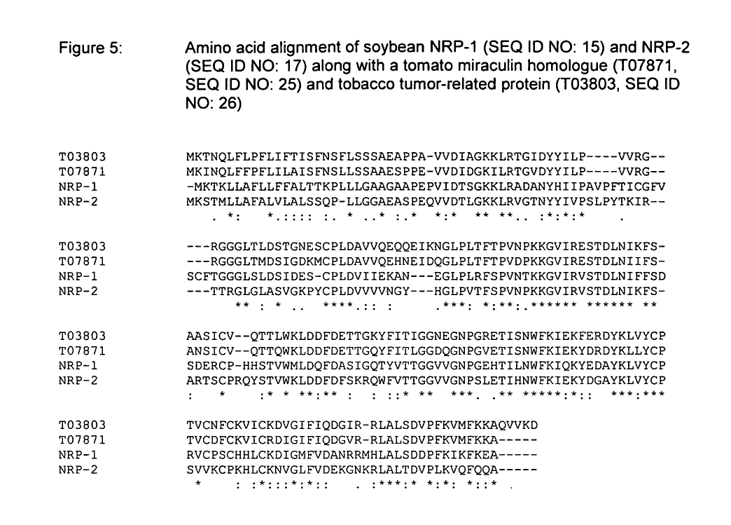

FIG. 5 shows the amino acid sequence alignment of soybean NRP-1 (SEQ ID NO: 15), NRP-2 (SEQ ID NO: 17), tomato miraculin homologue (T07871), and tobacco tumor-related protein (T03803).

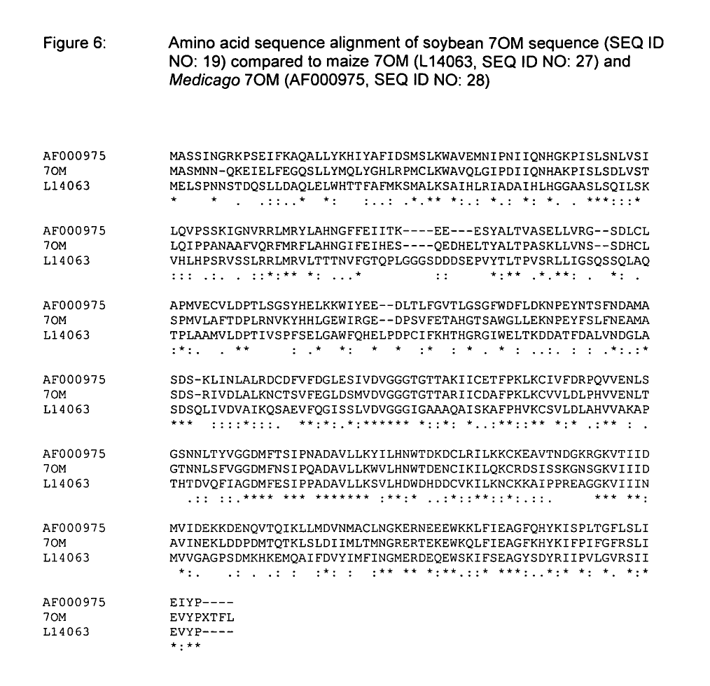

FIG. 6 is the amino acid sequence alignment of soybean and other plant 7OM proteins. The soybean 7OM protein (SEQ ID NO: 19) has 51% similarity and 40% identity with a maize 7OM homologue (L14063), and 68% similarity and 58% identity with a Medicago 7OM homologue (AF000975).

Down-regulation of Caffeoyl-coenzyme A O-methyltransferase (CCoAOMT) decreases the level of lignin, which is an important defense component. The suppression of CCoAOMT may also increase vanillic acid production. Vanillic acid is a sex pheromone isolated and identified from Heterodera glycines that induces male coiling needed for fertilization (Huettel and Rebois, (1986) J. Nematol. 18:3-8). Meyer and Hueftel ((1997). J. Nematol 29:282-288) hypothesized that addition of excessive amounts of vanillic acid to soil would substantially disrupt the SCN life cycle. Therefore, increase in vanillic acid levels may confer SCN resistance.

IPP plays an important role in signal transduction pathways, as discussed earlier in the instant specification. The soybean IPP was significantly induced by SCN infection (Table 1). Therefore soybean IPP has potential involvement in nematode-induced signals that lead to formation of the syncytium. Up- or down-regulation of the IPP gene may disrupt the formation of the syncytium and nematode development.

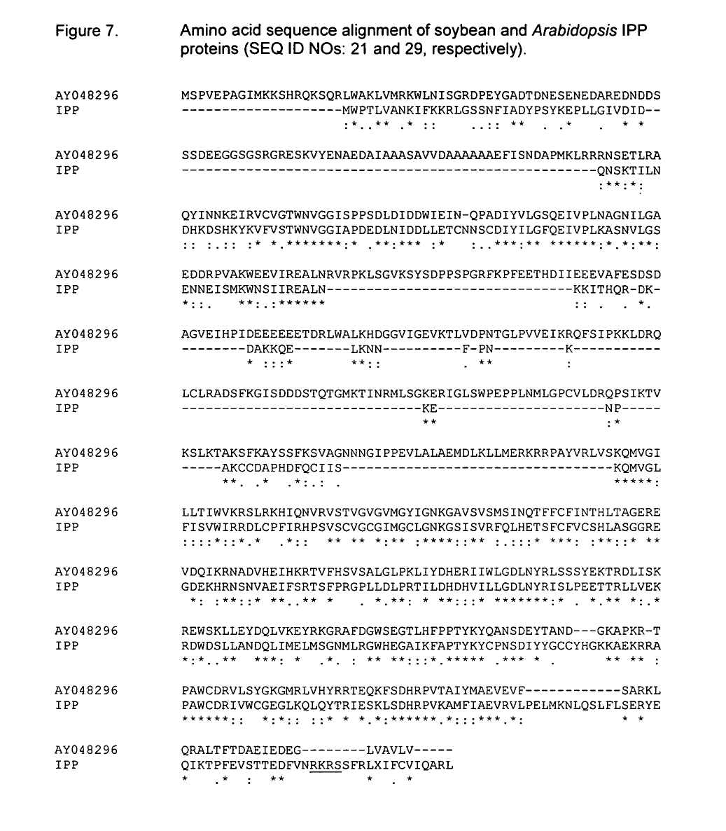

FIG. 7 is the amino acid sequence alignment of soybean and Arabidopsis IPP protein. There is 57% similarity and 46% identity between the soybean and Arabidopsis IPP. The potential CAMP and cGMP-dependent protein phosphorylation site is underlined.

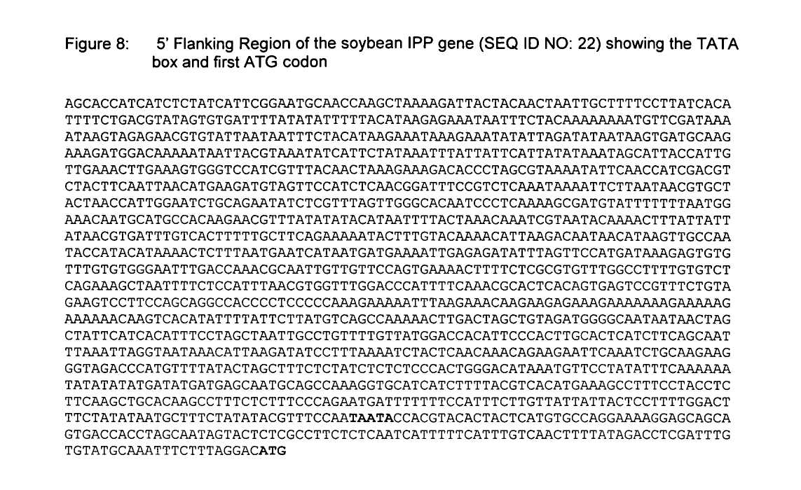

FIG. 8 shows the 5′-flanking region of the IPP gene. The first MET codon and potential TATA box were bolded.

IPP gene expression was significantly induced in soybean roots by SCN infection (Table 1). A soybean EST data base search using Tissue-Library Browser indicated that this IPP gene is root-specific. Therefore the IPP promoter is a potential root-specific and nematode-inducible promoter. We can use this promoter to specifically express nematode resistance genes to engineer nematode resistance in plants. The promoter contains potential nematode-responsive cis-acting elements that can be used to modify and generate nematode-regulated promoters.

### Example 4

### Production and Assay of Transformed Soybean Root Cultures

Agrobacterium rhizogenes strain K599 is used for soybean hairy root transformation, and the gene function and promoter activity are analyzed in transgenic soybean hairy roots. Stocks of A. rhizogenes are maintained on minimal A media (see recipes, below). Plasmid DNA is introduced into A. rhizogenes strain K599 using the freeze-thaw method, as described in Ha (1988) Plant Molecular Manual, eds. Gelvin, Schilperoort, and Verma, pp. A3/1-A3/7.

Soybean seeds are surface-sterilized with chlorine gas at room temperature for 12-16 hours. The seeds re then aerated in a clean air hood for at least 30 minutes. Seeds are germinated and cultured in Magenta™ boxes (Magenta Corporation) containing sterile potting soil with 10 to 15 mL of 25% Gamborg's B-5 Basal medium with minimal organics (G5893, Sigma). The boxes are placed under a mix of fluorescent and incandescent lights providing a 16-hour day/8-hour night cycle and constant temperature of about 26° C. Six-day-old seedlings of non-transformed plants are inoculated with a freshly grown culture of A. rhizogenes previously transformed with DNA constructs. The transformed A. rhizogenes is introduced into the hypocotyls just under the cotyledons by wounding 4 to 6 times in the epidermal cell layer with a 23 gauge needle containing the A. rhizogenes. The inoculated plants are cultured under the same conditions as those described above for seed germination.

After the soybean hypocotyls are inoculated with A. rhizogenes, adventitious soybean roots developed and were excised. Initially these putative transformed roots are cultured in liquid B-5 medium with antibiotics to cure the roots of any bacteria; antibiotics included 500 mg/L cefotaxime (Calbiochem-Novabiochem, La Jolla, Calif.) and 200 mg/L vancomycin (Spectrum Quality Products, Los Angeles, Calif.). Roots are transferred to fresh liquid medium every 2-3 days; this transfer to fresh media is performed a total of three times. After the third transfer, each root is moved to a plate of MXB medium with Gelrite™ gelling agent. To determine whether roots have been transformed, a 1-2 cm root piece is placed in a 1.5 mL tube with GUS staining solution (0.05% X-Gluc in 100 mM sodium phosphate buffer at pH 7.0 containing 10 MM EDTA, 0.1% Triton, and 0.5 mM K₄Fe(CN)-61420). Roots are incubated in this solution for 2 to 4 hours at 27 to 29° C.; solutions are then evaluated for development of the blue color indicative of GUS activity. Roots testing positive by this assay and control roots that are not transformed are cultured in MXB medium with Gelrite™ gelling agent in an incubator without light at 26 to 30° C. A 1-4 cm piece of root tip is excised and transferred to fresh medium every 2-4 weeks.

Roots testing positive for transformation with the DNA construct are assayed for resistance to infection by soybean cyst nematode (“SCN”). Roots are transferred to 6-well plates containing NM medium with Daishiin agar. After 4-10 days, roots are inoculated with second-stage SCN juveniles. Two to five root tips are placed in each well of a 6-well culture dish; four of the wells contain roots transformed with A. rhizogenes containing the DNA construct and the other two wells contain control roots transformed with A. rhizogenes not containing the DNA construct. One sample of control roots in this assay is an SCN-compatible control root sample from an SCN-susceptible or “compatible” soybean genotype such as Pioneer brand 9204. The other sample of control roots is an SCN-resistant soybean genotype such as Jack and is thus an SCN-resistant or “incompatible” control sample. Roots are inoculated by placing 500 second-stage SCN race 3 juveniles directly onto the roots in each well and incubating for 7 days at 26 to 28° C.

The following stock solutions and media are used for transformation and regeneration of soybean roots:

Stock Solutions (per Liter):

B-5 Majors: 25.00 g KNO₃, 1.34 g (NH₄)₂SO₄, 2.50 g MgSO₄-7H₂O, 1.50 g CaCl₂-2H₂O, 1.31 g NaH₂PO₄ (anhydrous).
B-5 Minors: 1.00 g MnSO₄-H₂O, 0.30 g H₃BO₃, 0.20 g ZnSO₄-7H₂O, 0.075 g KI.
B-5 Vitamin Stock with Thiamine: 1 L Vitamin B-5 Stock, 1 g Thiamine HCl.
Iron Mix: 3.73 g. Na₂EDTA, 2.78 g FeSO₄-7H₂O.
   Media (per Liter):
Minimal A medium: 10.5 g K₂HPO₄, 4.5 g KH₂PO₄, 1.0 g (NH₄)₂SO₄, 0.5 g (Na)₂C₆H₅O₇-2H₂O, 1 mL 1.0 M MgSO₄-7H₂O, 10 mL 20% w/v sucrose, 15 g agar,
B-5 medium: 0.6 g MES (2-(N-Morpholino) ethane-sulfonic acid (M5287, Sigma)), 20 g sucrose, 10 mL B-5 minors, 100 mL B-5 majors, 10 mL B-5 Vitamin Stock with Thiamine, 10 mL Iron mix.
MXB medium: Murashige and Skoog Basal nutrient salts (M5524, Sigma), 10 mL Vitamin B-5 Stock with Thiamine, 30 g sucrose.
MXB medium with Gelrite: add 3 g Gelrite™ gelling agent to 1 L MXB medium, pH 5.7.
MXB medium with Daishiin agar: add 8 g Daishiin agar to 1 L MXB medium, pH 6.5.

Histochemical Analysis of GUS Expression in SCN Syncytium

Root samples were infected with SCN and collected at different time points after inoculation. These samples were fixed in 0.1% glutaraldehyde in 25 MM phosphate buffer and infiltrated using a vacuum at 15 psi for 2 min. After washing in 25 mM phosphate buffer, root samples were immersed in GUS staining solution (0.05% 5-bromo-4-4chloroindolyl-(3-D-glucuronide in 100 mM sodium phosphate buffer, pH 7.0, containing 10 mM EDTA, 0.1% Triton, and 0.5 mM K₄Fe(CN)-6H₂O) and infiltrated for 2 min at 15 psi. The GUS staining was continued at 37° C. for 12 hours. Root samples were then boiled in acid fuschin solution for 2 minutes and destained in acidic glycerin (100 mL of glycerin and 20 μL of HCl). Samples were examined under a dissecting microscope for SCN-hairy root interaction and GUS expression patterns. Dissected root segments to be used for thin sectioning were fixed in 3% glutaraldehyde in 25 mM phosphate buffer for 2 hours and washed three times in 25 mM phosphate buffer for 30 min. Two different sectioning methods were used to prepare sections. The first method involved a three-step buffer exchange to replace ethanol with L.R. white resin (3:1; 1:1; and 1:3, ethanol 100: L.R. white resin). Roots were thin-sectioned (2 μm) with a Leica™ microtome and examined under a microscope. In a second method, root tissues were dehydrated through an ethanol series of 30%, 50%, 70% 95%, and three changes in 100% ethanol, with a 30-minute incubation per change. A gradual buffer exchange was then carried out to replace ethanol with Histoclear (100%) and then paraffin at 60° C. Roots were thin-sectioned (10 μm) with a Leica™ microtome, and whole root samples or thin sections were examined under dissecting and light microscopy.

### Example 5

### Soybean Embryo Transformation

To induce somatic embryos, cotyledons, 3-5 mm in length, dissected from surface sterilized, immature seeds of the soybean cultivar A2872, were cultured in the light or dark at 26° C. on an appropriate agar medium for six to ten weeks. Somatic embryos producing secondary embryos were then excised and placed into a suitable liquid medium. After repeated selection for clusters of somatic embryos that multiplied as early, globular staged embryos, the suspensions were maintained as described below.

Soybean embryogenic suspension cultures were maintained in 35 mL liquid media on a rotary shaker at 150 rpm and 26° C. with fluorescent lights on a 16:8 hour day/night schedule. Cultures were subcultured every two weeks by inoculating approximately 35 mg of tissue into 35 mL of liquid medium.

Soybean embryogenic suspension cultures were then transformed by the method of particle gun bombardment (Klein et al. (1987) Nature (London) 327:70-73, U.S. Pat. No. 4,945,050). A DuPont® Biolistic PDS 100/HE instrument (helium retrofit) was used for these transformations.

A selectable marker gene used to facilitate soybean transformation a transgene composed of the 35S promoter from Cauliflower Mosaic Virus (Odell et al. (1985) Nature 313: 810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the pyk20 gene operably linked to a synthetic promoter comprising pyk20 promoter sequences was isolated as a restriction fragment. This fragment was then inserted into a unique restriction site of the vector carrying the marker gene.

To 50 μL of a 60 mg/mL 1 μm gold particle suspension is added (in order): 5 μL DNA (1 μg/pL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL70% ethanol and resuspended in 40 μL of anhydrous ethanol. The DNA/particle suspension can be sonicated three times for one second each. Five microliters of the DNA-coated gold particles are then loaded on each macro carrier disk. Approximately 300-400 mg of a two-week-old suspension culture is placed in an empty 60×15 mm Petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/mL hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Antifungal Assay Protocols

Culture Maintenance:

Silica gel stocks of each fungus (Fusarium verticillioides MO33 isolate (hereinafter FVE), Fusarium graminearum 73B ISU isolate (hereinafter FGR) and Colletotrichum graminicola Carroll-IA-99 isolate (hereinafter CGR)) were prepared stored at −20° C.

Preparation of Cultures for Spore Production:

It is important to minimize the time that the silica gel-stored fungus is out of the −20° C. freezer, so all procedures should be performed as quickly as possible.

½ X potato dextrose agar plates are used for culturing FVE and CGR. 1/2 X oatmeal agar plates are used for culturing FGR. Each tube is flamed and about 5 crystals of the silica gel-stored fungus is sprinkled onto the agar surface. Two or three plates of each isolate are prepared. Each tube is then flamed again and sealed with its cap. Fresh lab film is reapplied and the tubes are returned to a −20° C. freezer. The newly plated cultures are stored in a plastic box (to prevent drying out) in the dark at room temperature.

New cultures of FGR and FVE should be started weekly and fresh CGR cultures should be started every other week in order to maintain a consistent supply of spores.

Spore Preparation:

For FGR and FVE, a portion of a 2 to 3 week old culture plate is rinsed with small amount of assay medium, which is then transferred to a sterile tube, vortexed and the spores are quantified using a hemacytometer.

For CGR, a sterile loop is gently dragged across orange areas of a 4 to 6 week old culture plate, and the orange spore mass should be visible on the loop. The loop is inserted into a small volume of assay media and mixed with the loop to suspend the spores. The spore mixture is then vortexed and the number of spores is quantified using a hemacytometer.

Spores are diluted to the desired concentration with assay medium (5,000 spores per mL for FGR and FVE, and 10,000 spores per mL for CGR). The spore solution is kept on ice not longer than 2 hours prior to the start of the assay

Assay Plate Preparation Details:

Standard non-tissue culture treated 96 well flat bottom plates are used for the assay as well as ½ area non-treated plates (Costar).

The assay medium used is ¼ X potato dextrose broth for FVE and FGR. ¼ X Czapec-Dox V8 is used for CGR. ¼ X CCM-phosphate may also be used to assay FGR & FVE. This more complete medium may be advantageous if assay results provide too many hits.

Media and spores are added to plates at a rate of 100 μL/well for a standard assay plate, or 50 μL/well for half area plates. When adding media and spores into plates that have had protein extracts dried into them, the pipette should be used repeatedly to withdraw and dispense the solution to re-suspend the dried protein. The plate is sealed with a gas permeable membrane (“Breathe-Easy”, Cat. No. BEM-1, Diversified Biotech, Boston, Mass.) and allowed to develop in the dark at 28° C.

After the incubation period, the plate is placed on an inverted microscope and each well is examined and scored on a scale of 0-4.0=no inhibition of fungal growth when compared to the negative control, 1=slight inhibition (overall growth is less than the negative control but growth from individual spores is not distinct), 2=moderate inhibition (growth from 1 spore can easily be identified and is significantly less abundant than the negative control), 3=strong inhibition (spores have germinated but growth is limited to a few branches of short hyphae), 4=complete inhibition (spores have not germinated). (See also: Duvick et al. 1992 J Biol Chem 267(26): 18814-18820)

Hits are defined as those samples that score 3 to 4 after a 24 hr incubation period. The concentration at which a sample achieves a score of 1 or higher is the minimal inhibitory concentration (MIC) and the concentration at which it achieves a score of 3 or higher is the minimal complete inhibitory concentration (MCIC). (See also Duvick et al. 1992 J Biol Chem 267(26): 18814-18820).

Media Recipes:

1× Czapek-Dox V8 Broth

For each liter suspend 35 g Difco Czapek-Dox Broth (#233810) in dH₂O and add 180 mL V8 juice that has been clarified by centrifugation (3,000×g is plenty). Raise final volume to 1 liter and autoclave at 121° C. for 20 minutes.

1X Potato Dextrose Broth

For each liter suspend 24 g Difco Potato Dextrose Broth (#0549-17-9) in dH₂O and raise final volume to 1 liter and autoclave at 121° C. for 20 minutes.

CCM (Cochliobolus Complete Medium)

Solution A: 10 grams Ca(NO₃)₂.4H₂O per 100 mL

Solution B: 2 grams KH₂PO₄+1.5 grams NaCl per 100 mL. Adjust pH to 5.3 with NaOH

Solution C: 2.5 grams MgSO₄.7H₂O per 100 mL

Put 900 mL dH₂O into vessel on stir plate. Add to the water in order and allow each component to dissolve before proceeding to the next step:
10 mL solution A
10 mL solution B
10 mL solution C
10 grams glucose
1 gram Difco yeast extract
0.5 gram casein hydrolysate (acid)
0.5 gram casein hydrolysate (enzyme)

Bring final volume to 1 liter and filter sterilize (do not autoclave).

¼ X CCM-phosphate is made by diluting the 1× CCM medium to ¼ X with 10 mM sodium phosphate buffer pH 5.8

¼ X potato dextrose agar

For each liter suspend 12 g Difco Potato Dextrose Broth (#0549-17-9) and 15 g agar in dH₂O, raise final volume to 1 liter and autoclave at 121° C. for 20 minutes. Pour plates in sterile hood.

Oatmeal Agar

For each liter suspend 36.24 g of Difco Oatmeal Agar (#0552-17-3) and 4.25 g agar in dH₂O in a 4 liter vessel. Cover and autoclave at 121° C. for 20 minutes. Pour plates in sterile hood.

TABLE 6
Conditions for fungal growth and sporulation

| | FVE | FGR | CGR |
|---|---|---|---|
| Isolate name | MO33 | 73B ISU | Carroll-IA-99 |
| Medium for | ½ × Potato | ½ × | ½ × Potato |
| sporulation | Dextrose Agar | Oatmeal Agar | Dextrose Agar |
| | (PDA) | | (PDA) |
| Agar culture age | 2-3 weeks old | 2-3 weeks old | 4-6 weeks old |
| range for in vitro | | | |
| assay | | | |
| Suggested | Weekly | Weekly | Every other week |
| schedule for | | | |
| starting agar | | | |
| cultures | | | |
| Liquid medium | ¼ × potato | ¼ × potato | ¼ × Czapec-Dox |
| for in vitro assay | dextrose broth | dextrose broth | V8 broth |
| Spore Density for | 5.00E+03 | 5.00E+03 | 1.00E+04 |
| in vitro assay | | | |
| (spores/mL) | | | |

### Example 7

### Soybean Cyst Nematode Infection Analysis of Transgenic Soybean Plants

Agrobacterium rhizogenes strain K599 was used for soybean hairy root transformation as described in Example 4 using the freeze thaw method.

T0 transgenic plants were transferred into cones with sterilized sand/soil mix (1:1 coarse sand and top soil), and grown for 3 weeks. The plants were then inoculated using SCN race 1 eggs as described below. Four to 6 plants per events were transplanted.

T1 and T2 seeds were directly planted in cones with sterilized sand/soil mix (1:1 coarse sand and top soil). Ten days after planting, the plants were inoculated with SCN race 1 eggs as described below. Ten plants per events were included in the T1 assays. Ten plants per line and 1-6 lines per event were included in T2 assays.

Susceptible check (Pioneer soybean line 93B87, 9281, and 9392), resistant check (Pioneer line 9234 and 92B12), and control plants (untransformed Jack, transformed Jack with Marker gene (hygromycin resistant gene) but no transgene) were planted in each experiment.

SCN race 1 females were harvested from fresh roots. Nematode inoculum was obtained from stock cultures of Lee 74 and P188788, which are grown together in clay pots. These plants were maintained in a greenhouse with soil temperatures of 70-84 degrees. Plants were allowed to grow 6 weeks to increase the population. A two gallon bucket was filled with two liters of tap water. Plant roots were gently rinsed in the tap water to remove as many of the soil particles as possible.

The roots were placed on a thoroughly cleaned 20 mesh sieve nested over a thoroughly cleaned 60 mesh sieve. The roots were gently rubbed on the 20 mesh sieve under a moderate stream of tap water (>20 seconds) to loosen the females. The roots were removed and the 20 mesh sieve nested over the 60 mesh sieve was rinsed for at least 10 seconds.

The 20 mesh sieve was removed, and the 60 mesh sieve was washed for at least 10 seconds with a moderate stream of tap water. The particles were collected on the edge of the 60 mesh sieve and rinsed into a clean container.

Floating and sinking materials were separated by density differentiation. The floating material was poured away by stirring the contents, letting the SCN females settle approximately 10 seconds and pouring away the lighter material. Then, the SCN females were poured away from the denser material by swirling the mixture and gently pouring the suspended SCN females over the 20/60 nested sieves. This step was repeated as many times as necessary.

When the SCN females were collected the final time on the 60 mesh sieve, they were washed into a clean container.

The females were rinsed into a small 60 mesh sieve and broken open with a motorized pestle set @ 70% for 3 minutes. The eggs were washed through the 200/500 mesh sieve and rinse into a clean container. The eggs were counted by placing one mL of solution on a glass slide under 20-60X magnification. The roots were then inoculated at about 10,000 eggs in less than 8 mL per water per plant.

Inoculation

Prior to inoculation, plants were selected (replicated for each line/variety) that were in a similar growth stage. A hole was created in the soil, close to the main stem of the plant, about ¾ inches deep and large enough to insert a pipette tip without it getting plugged or dirty. Each plant was inoculated with an equal quantity of eggs (constantly stirring the inoculum solution), using a pipette or a peristaltic pump. The inoculation site was closed with soil after inoculation.

Soil temperatures were checked each day. Soil temperature should be maintained at 70-84 F (76 F is optimum). Day length was set at 16 hours.

Cysts developed on the infected roots were counted four weeks after inoculation. Appropriate magnifiers and lighting were used to provide the best possible conditions for counting and scoring soybean roots.

The appropriate cone was removed from the planting tray, turned onto its side, and the plant is removed by one hard tap of the cone into the edge of a table, tray or reader's hand. A cotton ball was inserted into the cone for drainage purposes. The scorer needed to look at the cotton ball also, due to the fact that often the root was attached and cysts were present.

The plant was shaken gently 1-2 times to remove a large portion of the soil. The entire root was viewed prior to removing any more sand/soil or counting. If there was too much soil remaining for counting/scoring, step 3 was repeated. Depending on material type, there were 1-3 roots in each cone. If there was more than 1 root, the scorer gently teased the roots apart for counting. The Score/counts were total cyst counts of all roots in a single cone.

Beginning at the top of the root, cysts were counted one at a time, until the bottom of the root was reached, at which point the plant was rotated. Counting continued until the entire root was checked for cysts. It was sometimes necessary on roots with low counts to remove more soil. Soil was gently brushed off the root by hand, paying special attention not to also remove cysts. Soil was brushed into the palm of the hand and checked for cysts.

SCN Race1 Bioassay of UCP3:CDPKa Transgenic Soybean

The CDPKa gene was evaluated in stably transformed soybean plants at T0 and T1 stages. As indicated in table 7, over-expression of CDPKa under the SCN-inducible promoter UCP3 increased SCN race 1 reproduction in T0 and T1 transgenic soybean plants by 311% (average of 11 events) and 25% (average of 8 events), respectively, when compared to T0 and T1 transformed control Jack plants. These results, shown in table 7, indicate that the soybean CDPKa gene can increase SCN susceptibility in soybean. Co-suppression of the CDPKa gene can therefore confer SCN resistance.

TABLE 7
The effects of soybean CDPKa expression on SCN race 1 reproduction in
T0 and T1 generation soybean plants

| | CDPKa (T0) | | | | CDPKa (T1) | | |
|---|---|---|---|---|---|---|---|
| | | Average | Standard | | | Average | Standard |
| Event ID | PCR | cysts | Error | Event ID | PCR | cysts | Error |
| TC-Jack | N | 14.8 | 12.0 | TC-JACK | N | 52 | 28 |
| 3283-3-1 | P | 49.2 | 38.8 | 3283-5-1 | P | 18 | 14 |
| 3283-3-2 | P | 55.4 | 63.7 | 3283-5-6 | P | 30.1 | 17 |
| 3283-4-1 | P | 19.4 | 24.2 | 3284-5-2 | P | 73 | 84 |
| 3283-4-2 | P | 60.3 | 64.8 | 3335-1-5 | P | 66 | 34 |
| 3283-5-2 | P | 132.8 | 171.4 | 3335-2-3 | P | 50 | 31 |
| 3283-5-3 | P | 33.6 | 22 | 3335-3-1 | P | 68 | 65 |
| 3283-5-4 | P | 95.7 | 78.4 | 3335-3-12 | P | 127 | 96 |
| 3283-5-5 | P | 67.7 | 73.7 | 3335-3-9 | P | 57 | 57 |
| 3284-1-1 | P | 9.5 | 13.8 | | | | |
| 3284-1-2 | P | 46.6 | 76.5 | | | | |
| 3284-1-3 | P | 100.5 | 69.3 | | | | |
| Control | | 15 | 12 | Control | | 52 | 28 |

| | | | |
|---|---|---|---|
| Transgenic | 61 | Transgenic | 65 |

| | | | |
|---|---|---|---|
| Increased | 311% | Increased | 25% |

SCN Race1 Bioassay of UCP3:CDPKb Transgenic Soybean

The CDPKb gene was evaluated in stably transformed soybean plants at T0 and T1 stages. As indicated in table 8, over-expression of CDPKb under the SCN-inducible promoter UCP3 reduced SCN race 1 reproduction in T0 and T1 transgenic soybean plants by 7% (average of 11 events) and 29% (average of 9 events), respectively, when compared to T0 and T1 transformed control Jack plants. These results, shown in table 8 indicate that the CDPKb gene can reduce SCN susceptibility in soybean.

TABLE 8
The effects of soybean CDPKb expression on SCN race 1 reproduction in
T0 and T1 generation soybean plants

| | CDPKb (T0) | | | | CDPKb(T1) | | |
|---|---|---|---|---|---|---|---|
| | | Average | Standard | | | Average | Standard |
| Event ID | PCR | Cysts | Error | Event ID | PCR | Cysts | Error |
| TC-JACK | N | 25.7 | 2.3 | TC-JACK | N | 52 | 28.2 |
| 3304-5-4 | P | 14.7 | 12.5 | 3304-1-1 | P | 35.6 | 22.1 |
| 3304-4-5 | P | 3.0 | 1.7 | 3304-1-2 | P | 25.6 | 20 |
| 3304-6-5 | P | 9.3 | 4.0 | 3304-4-1 | P | 29.6 | 29.7 |
| 3304-2-2 | P | 2.0 | 0 | 3304-4-3 | P | 43.2 | 41.4 |
| 3304-3-3 | P | 28.5 | 12.0 | 3304-4-5 | P | 39 | 30.3 |
| 3304-4-4 | P | 33.5 | 9.2 | 3304-5-3 | P | 28.3 | 19.1 |
| 3304-4-1 | P | 40.3 | 14.6 | 3304-5-4 | P | 65.3 | 65 |
| 3304-1-2 | P | 33.3 | 16.3 | 3304-6-2 | P | 21.5 | 30.4 |
| 3304-5-3 | P | 29.3 | 5.9 | 3304-6-5 | P | 45.1 | 33.4 |
| 3304-6-3 | P | 31.5 | 9.2 | | | | |
| Control | | 25.7 | 2.3 | Control | | 52 | 28.2 |
| Trans- | 24.0 | | | Trans- | 37 | | |
| genics | | | | genics | | | |

| | | | |
|---|---|---|---|
| Reduced | 7% | Reduced | 29% |

SCN Race1 Bioassay of SCP1:AMPD Transgenic Soybeans

The AMPD gene was evaluated in stably transformed soybean plants at T1 stages. As indicated in table 9, over-expression of AMPD gene (AMP deaminase) under SCP1 promoter increased SCN race 1 reproduction in T1 transgenic soybean plants by 82% (average of 18 events) when compared to T1 transformed control Jack plants. These results, shown in table 12, indicate that the AMPD gene has an impact on SCN reproduction in soybean. Co-suppression of the AMPD gene can confer nematode resistance.

TABLE 9
The effects of soybean AMPD expression on SCN race 1 reproduction
in T1 generation soybean plants
AMPD (T1)

| | | | Standard | |
|---|---|---|---|---|
| Event ID | PCR | Average Cysts | Error | |
| TCJACK | N | 18.3 | 18.2 | |
| 3373.1.2 | P | 14.5 | 14.6 | |
| 3373.2.6 | P | 47.2 | 46.3 | |
| 3373.2.8 | P | 12.3 | 12.9 | |
| 3373.3.2 | P | 37.0 | 28.0 | |
| 3373.4.1 | P | 11.0 | 0.0 | |
| 3373.4.4 | P | 60.0 | 0.0 | |
| 3373.4.6 | P | 13.9 | 7.3 | |
| 3373.4.7 | P | 15.7 | 7.5 | |
| 3373.5.2 | P | 63.0 | 0.0 | |
| 3373.5.4 | P | 25.5 | 35.2 | |
| 3373.6.1 | P | 13.7 | 11.4 | |
| 3377.2.1 | P | 47.5 | 43.7 | |
| 3377.2.3 | P | 25.7 | 23.2 | |
| 3377.4.2 | P | 78.0 | 33.5 | |
| 3377.5.1 | P | 26.5 | 11.6 | |
| 3377.5.4 | P | 19.6 | 16.8 | |
| 3377.5.5 | P | 63.8 | 50.9 | |
| 3377.5.6 | P | 54.2 | 42.7 | |
| Control | | 18.3 | 18.2 | |
| Transgenics | | 33.3 | | |
| Increased | | | | 82% |

C) SCN Race1 Bioassay of UCP3:NRTF1a Transgenic Soybean

We evaluated the NRTF1a gene in stably transformed soybean plants at T0 and T1 stages. As indicated in table 10, over-expression of NRTF1a (a soybean AP2-like nematode-responsive transcription factor gene) under the SCN-inducible promoter UCP3, reduced SCN race 1 reproduction in T0 and T1 transgenic soybean plants by 42% (average of 13 events) and 40% (average of 8 events), respectively, when compared to T0 and T1 transformed control Jack plants. These results, shown in table 10, indicate that the. NRTF1a gene can confer resistance to SCN in soybean.

TABLE 10
The effects of soybean NRTF1a expression on SCN race 1 reproduction
in T0 and T1 generation soybean plants

| | NRTF1a(T0) | | | | NRTF1a (T1) | | |
|---|---|---|---|---|---|---|---|
| | | Average | Standard | | | Average | Standard |
| Event ID | PCR | Cysts | Error | Event ID | PCR | Cysts | Error |
| TC-JACK | N | 40 | 25 | TC-Jack | N | 52 | 28 |
| 3269-1-7 | P | 18 | 13 | 3269.2.1 | P | 36 | 33 |
| 3269-3-1 | P | 26 | 7 | 3269.3.6 | P | 32 | 19 |
| 3269-5-4 | P | 22 | 6 | 3269.5.2 | P | 38 | 14 |
| 3269-6-2 | P | 26 | 3 | 3269-6.4 | P | 47 | 23 |
| 3269-1-5 | P | 25 | 3 | 3269.6.9 | P | 30 | 18 |
| 3269-3-3 | P | 14 | 27 | 3269.3.1 | P | 28 | 20 |
| 3269-6-7 | P | 35 | 8 | 3269.3.2 | P | 20 | 14 |
| 3269-6-5 | P | 16 | 12 | 3270.6.1 | P | 16 | 9 |
| 3269-4-2 | P | 24 | 4 | | | | |
| 3269-2-2 | P | 10 | 3 | | | | |
| 3269-1-2 | P | 20 | 21 | | | | |
| 3269-1-4 | P | 31 | 22 | | | | |
| 3269-6-3 | P | 34 | 25 | | | | |
| Control | | 40 | 25 | Control | | 52 | 28 |

| | | | |
|---|---|---|---|
| Transgenic | 23 | Transgenic | 31 |

| | | | |
|---|---|---|---|
| Reduced | 42% | Reduced | 40% |

D) SCN Race1 Bioassay of SCP1:NRP-1 Transgenic Soybean

The NRP gene was evaluated in stably transformed soybean plants at T0 and T1 stages. As indicated in table 11, over-expression of NRP under the SCP1 promoter reduced SCN race 1 reproduction in T1 and T1 transgenic soybean plants by 26% (average of 11 events) and 45% (average of 14 events), respectively, when compared to T0 and T1 transformed control Jack plants. These results, shown in table 11, indicate that the NRP genes of the invention can reduce SCN susceptibility in soybean.

TABLE 11
The effects of soybean NRP-1 expression on SCN race 1 reproduction in
T0 and T1 generation soybean plants

| | NRP-1(T0) | | | | | NRP-1(T1) | | | |
|---|---|---|---|---|---|---|---|---|---|
| | | Average | Standard | | | | Average | Standard | |
| Event ID | PCR | Cysts | Error | | Event ID | PCR | Cysts | Error | |
| TC_Jack | N | 25.7 | 2.3 | | TC-Jack | N | 52 | 28.2 | |
| 3314-3-3 | P | 9.0 | 2.6 | | 3250-4-2 | P | 18.8 | 8.7 | |
| 3314-2-5 | P | 9.7 | 2.8 | | 3250-4-3 | P | 47.3 | 24.9 | |
| 3314-3-2 | P | 6.3 | 5.1 | | 3250-5-1 | P | 13.4 | 13 | |
| 3314-2-3 | P | 2.0 | 0 | | 3250-5-3 | P | 17.7 | 15.5 | |
| 3314-2-2 | P | 12.7 | 10.3 | | 3250-5-5 | P | 24.7 | 14 | |
| 3314-1-1 | P | 13.5 | 7.2 | | 3250-5-6 | P | 35.1 | 29.2 | |
| 3314-2-1 | P | 19.5 | 4.9 | | 3250-6-6 | P | 62.1 | 37.6 | |
| 3314-2-6 | P | 34.3 | 3.5 | | 3250-6-7 | P | 24.0 | 17.4 | |
| 3314-2-9 | P | 37.0 | 8.5 | | 3314-1-2 | P | 14.2 | 14.6 | |
| 3314-2-6 | P | 30.5 | 7.8 | | 3314-2-3 | P | 37.5 | 21.9 | |
| 3314-6-1 | P | 34.7 | 23.7 | | 3314-2-4 | P | 32.1 | 31.6 | |
| | | | | | 3314-2-5 | P | 31.5 | 26.3 | |
| | | | | | 3314-2-7 | P | 11.0 | 5.6 | |
| | | | | | 3314-6-2 | P | 28.2 | 22.6 | |
| Control | | 25.7 | 2.3 | | Control | | 52 | 28.2 | |
| Transgenics | | 19.0 | | | Transgenics | | 28.4 | | |
| Reduced | | | | 26% | Reduced | | | | 45% |

E) SCN Race1 Bioassay of SCP1:7OM Transgenic Soybeans

The 7OM (7-O-methyltransferase) gene was evaluated in stably transformed soybean plants at T1 stages. As indicated in table 12, over-expression of 7OM gene under the SCP1 promoter increased SCN race 1 reproduction in T1 transgenic soybean plants by 71% (average of 16 events) when compared to T1 transformed control Jack plants. These results, shown in table 12 indicate that the 7OM gene has an impact on SCN reproduction in soybean. Co -suppression of the 7OM gene will confer SCN resistance.

TABLE 12
The effects of soybean 7OM expression on SCN race 1 reproduction
in T1 generation soybean plants

| 7OM (T1) | | | | |
|---|---|---|---|---|
| Event ID | PCR | Average Cysts | Standard Error | |
| TCJACK | N | 12.4 | 10.9 | |
| 3361.2.3 | P | 17.6 | 16.7 | |
| 3361.3.2 | P | 35.2 | 29.5 | |
| 3361.3.6 | P | 14.6 | 15.2 | |
| 3361.4.1 | P | 12.4 | 10.8 | |
| 3361.5.2 | P | 26.0 | 25.7 | |
| 3365.1.2 | P | 39.1 | 30.7 | |
| 3365.2.1 | P | 18.5 | 14.8 | |
| 3365.2.2 | P | 16.8 | 12.2 | |
| 3365.2.3 | P | 37.2 | 25.7 | |
| 3365.2.4 | P | 11.2 | 7.2 | |
| 3365.2.5 | P | 29.7 | 21.2 | |
| 3365.2.6 | P | 11.6 | 9.6 | |
| 3365.2.7 | P | 27.6 | 12.0 | |
| 3365.3.4 | P | 15.0 | 6.8 | |
| 3365.4.1 | P | 10.8 | 7.3 | |
| 3365.4.2 | P | 16.0 | 11.2 | |
| Control | | 12.4 | 10.9 | |
| Transgenics | | 21.2 | | |
| Increased (%) | | | | 71% |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims. Thus, many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims.

1. An isolated nucleic acid molecule, said molecule encoding a nematode-responsive protein wherein said nucleic acid molecule comprises a nucleotide sequence selected from the group consisting of:
(a) a nucleotide sequence comprising the sequence set forth in SEQ ID NOs: 1, 3, 6, 8, 10, 12, 14, 16, 18, or 20;
(b) a nucleotide sequence comprising an effective number of contiguous nucleotides of the sequence of SEQ ID NOs: 1, 3, 6, 8, 10, 12, 14, 16, 18, or 20 so that said nucleotide sequence encodes a protein that retains nematode-responsive activity;
(c) a nucleotide sequence having at least 90% sequence identity to the sequence of SEQ ID NOs: 1, 3, 6, 8, 10, 12, 14, 16, 18, or 20, wherein said nucleotide sequence encodes a protein with nematode-responsive activity;
(d) a nucleotide sequence which hybridizes under conditions of high stringency to SEQ ID NOs: 1, 3, 6, 8, 10, 12, 14, 16, 18, or 20, wherein said nucleotide sequence encodes a protein with nematode-responsive activity;
(e) a nucleic acid molecule comprising a sequence encoding the amino acid sequence set forth in SEQ ID NOs: 2, 4, 7, 9, 11, 13, 15, 17, 19 or 21; and
a nucleic acid molecule comprising a sequence deposited as ATCC Deposit No. PTA-4153.

2. The isolated nucleic acid molecule of claim 1, wherein said isolated nucleic acid molecule encodes a polypeptide having CDPK, NRTF1, NRP, 7OM or IPP activity.

3. An expression cassette comprising the nucleic acid molecule of claim 1.

4. The expression cassette of claim 3, wherein said nucleic acid molecule is operably linked to a promoter that drives expression in a host cell.

5. A plant cell having stably incorporated in its genome the nucleic acid molecule of claim 1.

6. A plant cell having stably incorporated into its genome at least one expression cassette of claim 3.

7. The plant cell of claim 6, wherein said plant cell is from a dicot plant.

8. The plant cell of claim 7, wherein said dicot plant is soybean.

9. The plant cell of claim 6, wherein said plant cell is a root cell.

10. A nucleic acid molecule that drives expression of an operably linked nucleic acid sequence in a plant cell, wherein said nucleic acid molecule comprises a nucleotide sequence selected from the group consisting of:
(a) a nucleotide sequence comprising the sequence set forth in SEQ ID NOs: 5 or 22;
(b) a nucleotide sequence comprising an effective number of contiguous nucleotides of the sequence of SEQ ID NOs: 5 or 22, wherein said nucleotide sequence regulates expression of an operably linked nucleic acid sequence in a nematode-responsive manner;
(c) a nucleotide sequence having at least 90% sequence identity to the sequence of SEQ ID NOs: 5 or 22, wherein said nucleotide sequence regulates expression of an operably linked nucleic acid sequence in a nematode-responsive manner; and
(d) a nucleotide sequence which hybridizes under conditions of high stringency to SEQ ID NOs: 5 or 22, wherein said nucleotide sequence regulates expression of an operably linked nucleic acid sequence in a nematode-responsive manner.

11. An expression cassette comprising the nucleic acid molecule of claim 10.

12. A plant cell having stably incorporated in its genome the nucleic acid molecule of claim 10.

13. The plant cell of claim 12, wherein the plant cell is from a dicot plant.

14. The plant cell of claim 13, wherein the dicot plant is soybean.

15. The plant cell of claim 12, wherein said cell is a root cell.

16. A method for inducing transcription in a plant cell, of an operably linked heterologous nucleic acid sequence, said method comprising transforming a plant cell with a nucleic acid molecule operably linked to a promoter that regulates transcription of said sequence in a plant cell in response to a nematode stimulus; wherein said promoter comprises a nucleotide sequence selected from the group consisting of:
(a) a nucleotide sequence comprising the sequence set forth in SEQ ID NOs: 5 or 22;
(b) a nucleotide sequence comprising an effective number of contiguous nucleotides of the sequence of SEQ ID NOs: 5 or 22, wherein said nucleotide sequence has nematode-regulated promoter activity;
(c) a nucleotide sequence having at least 90% sequence identity to the sequence of SEQ ID NOs: 5 or 22, wherein said nucleotide sequence has nematode-regulated promoter activity; and
(d) a nucleotide sequence which hybridizes under conditions of high stringency to SEQ ID NOs: 5 or 22, wherein said nucleotide sequence has nematode-regulated promoter activity.

17. A method of modulating the expression of a nucleotide sequence of interest in a plant, said method comprising:
(a) transforming a plant cell with a nucleic acid molecule comprising said nucleotide sequence of interest operably linked to a promoter which induces transcription of said sequence in a plant cell in response to a nematode stimulus, wherein said promoter is selected from the group consisting of:
(i) a nucleotide sequence comprising the sequence set forth in SEQ ID NOs: 5 or 22;
(ii) a nucleotide sequence comprising an effective number of contiguous nucleotides of the sequence of SEQ ID NOs: 5 or 22, wherein said nucleotide sequence has nematode-regulated promoter activity;
(iii) a nucleotide sequence having at least 90% sequence identity to the sequence of SEQ ID NOs: 5 or 22, wherein said nucleotide sequence has nematode-regulated promoter activity; and
(iv) a nucleotide sequence which hybridizes under conditions of high stringency to SEQ ID NOs: 5 or 22, wherein said nucleotide sequence has nematode-regulated promoter activity.
(b) regenerating a stably transformed plant from said plant cell; and
(c) exposing said plant to said nematode stimulus.

18. The method of claim 17, wherein said plant is a dicot.

19. The method of claim 18, wherein said dicot is soybean.

20. The method of claim 17, wherein expression is altered in the root tissues of said plant, wherein said root tissues are selected from the group consisting of pericycle and vascular cylinder.

21. A plant stably transformed with a nucleic acid molecule comprising a heterologous nematode-responsive sequence operably linked to a promoter that induces transcription of said nematode-responsive sequence in a plant cell in response to a nematode stimulus, wherein said promoter comprises a nucleotide sequence selected from the group consisting of:
(a) a nucleotide sequence comprising the sequence set forth in SEQ ID NOs: 5 or 22;
(b) a nucleotide sequence comprising an effective number of contiguous nucleotides of the sequence of SEQ ID NOs: 5 or 22, wherein said nucleotide sequence has nematode-regulated promoter activity;
(c) a nucleotide sequence having at least 90% sequence identity to the sequence of SEQ ID NOs: 5 or 22, wherein said nucleotide sequence has nematode-regulated promoter activity; and
(d) a nucleotide sequence which hybridizes under conditions of high stringency to SEQ ID NOs: 5 or 22, wherein said nucleotide sequence has nematode-regulated promoter activity.

22. The plant of claim 21, wherein said plant is a dicot.

23. The plant of claim 21, wherein said dicot is soybean.

24. Transformed seed of any of the plants of claims 21-23, wherein the seed comprise the nucleotide sequence.

25. A method for conferring or improving nematode resistance in a plant, said method comprising:
(a) transforming said plant with a nucleic acid molecule comprising a heterologous sequence operably linked to a regulatory sequence that induces transcription of said heterologous sequence in a plant cell in response to a nematode stimulus; and
(b) regenerating stably transformed plants, wherein said heterologous sequence comprises a nucleotide sequence selected from the group consisting of:
(i) a nucleotide sequence comprising the sequence set forth in SEQ ID NOs: 1, 3, 6, 8, 10, 12, 14, 16, 18, 20 or 33;
(ii) a nucleotide sequence comprising an effective number of contiguous nucleotides of the sequence of SEQ ID NOs: 1, 3, 6, 8, 10, 12, 14, 16, 18, 20 or 33, wherein said nucleotide sequence encodes a protein with nematode-responsive activity;
(iii) a nucleotide sequence having at least 90% sequence identity to the sequence of SEQ ID NOs: 1, 3, 6, 8, 10, 12, 14, 16, 18, 20 or 33, wherein said nucleotide sequence encodes a protein with nematode-responsive activity; and
(iv) a nucleotide sequence which hybridizes under conditions of high stringency to SEQ ID NOs: 1, 3, 6, 8, 10, 12, 14,16, 18, 20 or 33, wherein said nucleotide sequence encodes a protein with nematode-responsive activity; and
(c) exposing said plant to said nematode stimulus.

26. A plant stably transformed with a nucleic acid molecule comprising a heterologous sequence operably linked to a regulatory sequence that induces transcription of said heterologous sequence in a plant cell in response to a nematode stimulus, wherein said heterologous sequence comprises a nucleotide sequence selected from the group consisting of:
(a) a nucleotide sequence comprising the sequence set forth in SEQ ID NOs: 1, 3, 6, 8, 10, 12, 14, 16, 18, 20 or 33;
(b) a nucleotide sequence comprising an effective number of contiguous nucleotides of the sequence of SEQ ID NOs: 1, 3, 6, 8, 10, 12, 14, 16, 18, 20 or 33, wherein said nucleotide sequence encodes a protein with nematode-responsive activity;
(c) a nucleotide sequence having at least 90% sequence identity to the sequence of SEQ ID NOs: 1, 3, 6, 8, 10, 12, 14, 16, 18, 20 or 33, wherein said nucleotide sequence encodes a protein with nematode-responsive activity; and
(d) a nucleotide sequence which hybridizes under conditions of high stringency to SEQ ID NOs: 1, 3, 6, 8, 10, 12, 14, 16, 18, 20 or 33, wherein said nucleotide sequence encodes a protein with nematode-responsive activity.

27. The plant of claim 26, wherein said promoter is selected from the group consisting of a constitutive promoter, a tissue-preferred promoter, and an inducible promoter.

28. The plant of claim 26, wherein said plant is a monocot.

29. The plant of claim 26, wherein said monocot is selected from the group consisting of maize, wheat, rice, barley, sorghum, and rye.

30. The plant of claim 26, wherein said plant is a dicot.

31. The dicot plant of claim 30, wherein said dicot plant is soybean.

32. Transformed seed of the plant of any of claims 26-31, wherein the seed comprise the heterologous nucleotide sequence.

33. An isolated polypeptide having nematode-regulated activity, wherein said polypeptide is selected from the group consisting of:
(a) a polypeptide comprising the amino acid sequence set forth in SEQ ID NOs: 2, 4, 7, 9, 11, 13, 15, 17, 19, or 21;
(b) a polypeptide encoded by a nucleotide sequence comprising the sequence set forth in SEQ ID NOs: 1, 3, 6, 8, 10, 12, 14, 16, 18, or 20;
(c) a polypeptide comprising an amino acid sequence encoded by a nucleotide sequence deposited as Deposit No. PTA-4153;
(d) a polypeptide encoded by a nucleotide sequence that has at least 90% sequence identity to the sequence set forth in SEQ ID NOs: 1, 3, 6, 8, 10, 12, 14, 16, 18, or 20;
(e) a polypeptide comprising an amino acid sequence having at least 90% sequence identity to the sequence set forth in SEQ ID NOs: 2, 4, 7, 9, 11, 13, 15, 17, 19, or 20; and
(f) a polypeptide comprising an effective number of contiguous amino acids of any of (a) through (e), wherein said polypeptide retains nematode-regulated activity.

34. A method for conferring or improving nematode resistance in a plant, said method comprising:
(a) transforming said plant with a nucleic acid construct designed to inhibit or suppress expression of a native sequence; and
(b) regenerating stably transformed plants, wherein said native sequence comprises a nucleotide sequence selected from the group consisting of:
(i) a nucleotide sequence comprising the sequence set forth in SEQ ID NOs: 1, 3, 6, 8, 10, 12, 14, 16, 18, 20, or 33;
(ii) a nucleotide sequence comprising an effective number of contiguous nucleotides of the sequence of SEQ ID NOs: 1, 3, 6, 8, 10, 12, 14, 16, 18, 20, or 33, wherein said nucleotide sequence encodes a protein with nematode-responsive activity; and
(iii) a nucleotide sequence having at least 90% sequence identity to the sequence of SEQ ID NOs: 1, 3, 6, 8, 10, 12, 14, 16, 18, 20, or 33, wherein said nucleotide sequence encodes a protein with nematode-responsive activity.
